(19) United States
(12) Patent Application Publication
(10) Pub. No.: US 2013/0324880 A1
(43) Pub. Date: Dec. 5, 2013

(54) AUDITORY EVENT-RELATED POTENTIAL MEASUREMENT SYSTEM, AUDITORY EVENT-RELATED POTENTIAL MEASUREMENT APPARATUS, AUDITORY EVENT-RELATED POTENTIAL MEASUREMENT METHOD, AND COMPUTER PROGRAM THEREOF

(71) Applicant: Panasonic Corporation, Osaka (JP)

(72) Inventors: Shinobu ADACHI, Nara (JP); Jun OZAWA, Nara (JP)

(73) Assignee: Panasonic Corporation, Osaka (JP)

(21) Appl. No.: 13/960,486

(22) Filed: Aug. 6, 2013

Related U.S. Application Data
(63) Continuation of application No. PCT/JP2012/006611, filed on Oct. 16, 2012.

(30) Foreign Application Priority Data
Oct. 18, 2011 (JP) 2011-228575

(51) Int. Cl. A61B 5/0484

(52) U.S. Cl. CPC A61B 5/04845
USPC 600/545

(57) ABSTRACT

The auditory event-related potential measurement system includes: a size determination section for determining a size of a region within a video to be presented to a user so that the region has a viewing angle between diagonal corners in a range greater than 2 degrees and smaller than 14 degrees; a video output section for presenting to the user a video including a region of the size determined by the size determination section; an auditory stimulation output section for presenting an auditory stimulation to the user during a period in which the video is being presented to the user; a biological signal measurement section for measuring an electroencephalogram signal of the user; and an electroencephalogram processing section for acquiring an event-related potential from the electroencephalogram signal as reckoned from a point in time at which the auditory stimulation is presented.

This is a continuation of International Application No. PCT/JP2012/006611, with an international filing date of Oct. 16, 2012, which claims priority of Japanese Patent Application No. 2011-228575, filed on Oct. 18, 2011, the contents of which are hereby incorporated by reference.

### 1. TECHNICAL FIELD

The present disclosure relates to a technique of measuring with a high accuracy an auditory event-related potential in response to an auditory stimulation. More specifically, the present disclosure relates to a method of presenting an auditory stimulation while presenting a video, and measuring an auditory event-related potential without being influenced by fluctuations in the arousal level of a user or the video.

### 2. DESCRIPTION OF THE RELATED ART

In recent years, due to the downsizing and improved performance of hearing aids, there is an increasing number of users of hearing aids. In accordance with the deteriorated state of hearing of each user, a hearing aid amplifies an audio signal of a frequency band in which his or her hearing has deteriorated, this amplification being adapted to the degree of hearing deterioration. This makes it easier for the user to hear sounds.

Since each user may have a different deteriorated state of hearing, it is necessary to correctly evaluate each user's hearing before beginning the use of a hearing aid. Then, based on that evaluation result, a “fitting” is performed to determine an amount of sound amplification for each frequency.

Generally speaking, hearing of each user is evaluated based on the user's subjective report. A subjective report is made by indicating the user's own evaluation as to whether a sound is heard to the user or not, either orally or by pressing a button, etc. However, evaluation through subjective reporting has problems in that the results will vary depending on the linguistic expression and personality, and that evaluation is impossible with infants who are unable to give subjective reports. Therefore, techniques of objectively evaluating hearing without relying on any subjective reporting are under development.

An electroencephalogram is an effective tool for measuring user states such as perception and cognition. An electroencephalogram, which reflects neural activities of the cerebral cortex, is obtained by recording potential changes between two points on the scalp. While recording an electroencephalogram through electrodes which are worn on the scalp of a user, an auditory stimulation is presented to the user, in response to which a characteristic electroencephalogram is induced based on the auditory stimulation as a starting point. This electroencephalogram is called an auditory event-related potential. An auditory event-related potential is an index which enables objective evaluation of a user's hearing. An auditory event-related potential contains an extrinsic component (auditory evoked potential) which is evoked by an auditory stimulation, as well as an intrinsic component caused by exposure to the auditory stimulation.

Hoppe, U., et al., “Loudness perception and late auditory evoked potentials in adult cochlear implant users”, 2001 (hereinafter referred to as “Non-Patent Document 1”) suggests a possibility of being able to identify a relationship between “loudness” (as a user's subjective index of perceived loudness) and the amplitude and latency of an N1 component in response to an auditory stimulation of a pure tone, and estimate a loudness, among other hearing evaluations, from the amplitude and latency of the N1 component. Note that an “N1 component” is a negative sensory evoked potential which is induced at about 100 ms based on the presentation of an auditory stimulation as a starting point. Since the N1 component reflects neural activities of the cerebral cortex, it is believed that the N1 component has a higher correlation with one's subjective perception than a brain stem response (ABR) does. This indicates a possibility that loudness, among other hearing evaluations, can be estimated from the amplitude and latency of the N1 component.

Mariam, M., et al., “Comparing the habituation of late auditory evoked potentials to loud and soft sound”, 2009, (hereinafter referred to as “Non-Patent Document 2”) discloses an uncomfortableness level estimation technique utilizing habituation of the N1 component. An “uncomfortableness level” (uncomfortable level: also referred to as “UCL” in the present specification) is a smallest sound pressure that is too loud to be heard for a long time. This technique utilizes the fact that habituation of the N1 component does not occur when a sound is so loud that it is unignorable.

Since an auditory event-related potential has a low signal-to-noise ratio (S/N) relative to the background electroencephalogram, it is necessary to reduce the influence of mixed noises by repetitively presenting the stimulation and taking an arithmetic mean. Therefore, given a number N of repetitions, an amount of time which is equal to N times the stimulation interval is needed. For example, in Non-Patent Document 2, where 800 times of repetition are made with a stimulation interval of 1 second, 800 seconds (i.e., ten and several minutes) are required for each kind of auditory stimulation.

### SUMMARY

In the aforementioned conventional techniques, there is a need to conduct quicker electroencephalogram measurement, and make more accurate hearing evaluations.

A non-limiting and illustrative embodiment of the present disclosure provides, in an auditory event-related potential measurement system for hearing evaluation, a technique of suppressing fluctuations in auditory event-related potential due to changes in the arousal level, and measuring an auditory event-related potential with a high accuracy.

In one general aspect, an auditory event-related potential measurement system disclosed herein includes: a size determination section configured to determine a size of a region within a video to be presented to a user so that the region has a viewing angle between diagonal corners in a range greater than 2 degrees and smaller than 14 degrees; a video output section configured to present to the user a video including a region of the size determined by the size determination section; an auditory stimulation output section configured to present an auditory stimulation to the user during a period in which the video is being presented to the user; a biological signal measurement section configured to measure an electroencephalogram signal of the user; and an electroencephalogram processing section configured to acquire an event-related potential from the electroencephalogram signal as reckoned from a point in time at which the auditory stimulation is presented.

According to the above aspect, fluctuations in auditory event-related potential due to changes in the arousal level of a user are reduced, whereby a highly accurate auditory event-related potential measurement can be realized.

These general and specific aspects may be implemented using a system, a method, and a computer program, or any combination of systems, methods, and computer programs.

Additional benefits and advantages of the disclosed embodiments will be apparent from the specification and Figures. The benefits and/or advantages may be individually provided by the various embodiments and features of the specification and drawings disclosure, and need not all be provided in order to obtain one or more of the same.

### BRIEF DESCRIPTION OF DRAWINGS

FIGS. 1A and 1B show an auditory event-related potential measurement paradigm where only auditory stimulations are used, and imaginary changes in arousal level during an auditory event-related potential measurement.

FIGS. 2A and 2B show an auditory event-related potential measurement paradigm where a video is concurrently presented, and imaginary changes in arousal level during an auditory event-related potential measurement.

FIG. 3 is a table showing subjectively-reported values of uncomfortable sound pressure obtained in a subjective report experiment conducted by the inventors.

FIG. 4 is a diagram showing an auditory stimulation combination used in an electroencephalographic experiment conducted by the inventors.

FIGS. 5A and 5B show electrode positions according to the International 10-20 system, and electrode positions in an electroencephalographic experiment conducted by the inventors.

FIG. 6 show characteristic data of event-related potential in an electroencephalographic experiment conducted by the inventors.

Portions (a) to (c) of FIG. 7 are graphs showing N1-P2 amplitude in response to first to third sounds, with respect to different frequencies.

FIG. 8 is a graph showing example wavelet coefficients of event-related potential in an electroencephalographic experiment conducted by the inventors.

FIG. 9 is a table showing an example of training data used in an uncomfortable sound pressure estimation conducted by the inventors.

FIG. 10 is a graph showing subjectively-reported values obtained from a subjective report experiment and fluctuation in results of an uncomfortable sound pressure estimation made from an electroencephalographic experiment.

FIG. 11 is a table showing conditions of an experiment which was conducted by the inventors for determining the influence of the size of video presentation on auditory event-related potential.

FIGS. 12A and 12B are bar charts showing results of subjective reporting of arousal levels in an experiment conducted by the inventors.

FIGS. 13A and 13B are bar charts showing results of subjective reporting of eye fatigue in an experiment conducted by the inventors.

FIG. 14 is a bar chart showing estimation errors with different sizes of video presentation in an experiment conducted by the inventors.

FIG. 15 is a diagram showing a construction and an environment of use for an auditory event-related potential measurement system 1 according to an illustrative embodiment.

FIG. 16 is a diagram showing the hardware construction of an auditory event-related potential measurement apparatus 10 according to an illustrative embodiment.

FIG. 17 is a diagram showing the functional block construction of an auditory event-related potential measurement system 1 according to an illustrative embodiment.

FIG. 18 is a flowchart showing a procedure of processing performed by the auditory event-related potential measurement system 1.

FIG. 19 is a diagram illustrating the definition of a viewing angle in the present specification.

FIGS. 20A and 20B are diagrams showing examples of determining a diagonal length (S) of an object for viewing angle calculation.

FIG. 21 is a diagram schematically showing a main region 201a whose size is changeable.

### DETAILED DESCRIPTION

In conventional techniques such as Non-Patent Document 1 and Non-Patent Document 2 above, a monotonous auditory stimulation is presented for a long time. For this reason, the user may often be unable to maintain his or her arousal level. As is stated in supervised by Sato et al., “BASIC AND CLINICAL EVOKED POTENTIAL”, p. 129, SOZO-SHUPPAN, 1990 (first edition), it is currently believed that the auditory event-related potential undergoes great changes in its waveform depending on the arousal level. Therefore, even when a hearing evaluation is made by the conventional techniques using the amplitude and latency of an N1 component, there is a possibility that the evaluation may not be correct.

Hereinafter, with reference to the attached drawings, embodiments of the auditory event-related potential measurement system according to the present disclosure will be described.

First, the terminology used in the present specification will be described.

An “event-related potential (event-related potential: ERP)” is a kind of electroencephalogram (electroencephalogram: EEG), and refers to a transient potential fluctuation of the brain that occurs in temporal relationship with an external or internal event.

An “auditory event-related potential” is an event-related potential that is induced in response to an auditory stimulation. Examples thereof are: a P1 component, which is a positive potential that is induced at about 50 ms since an auditory stimulation as a starting point; an N1 component, which is a negative potential that is induced at about 100 ms since an auditory stimulation as a starting point; and a P2 component, which is a positive potential that is induced at about 200 ms since an auditory stimulation as a starting point.

To “present a sound” means outputting an auditory stimulation of a pure tone, e.g., outputting a pure tone through one ear of headphones.

A “pure tone” is a sound, repeating its periodic oscillation, that is expressed by a sine wave having only one frequency component. The type of headphones for presenting pure tones may be arbitrary, so long as the headphones are able to accurately output a pure tone with a designated sound pressure. This makes it possible to correctly measure an uncomfortable sound pressure.

An “electrooculogram (EOG)” is a potential fluctuation which is induced by an eye movement. An electrooculogram occurs due to electrical charging of an eyeball. The cornea of an eyeball has a plus charge, whereas the retina has a minus charge. As an eye movement changes the electrical charges of the cornea and the retina, the skin around the eye undergoes a change in potential; this potential change in the skin is detected as an electrooculogram. The amplitude of an electrooculogram may be about several dozen times the event-related potential, even at an electrode on the scalp. An electrooculogram may become a noise in the event-related potential.

A “viewing angle” is an angle constituted by an object which is projected onto the eye. In the present specification, θ satisfying eq. 1 below is detected as a viewing angle.

tan θ=S/D (eq. 1)

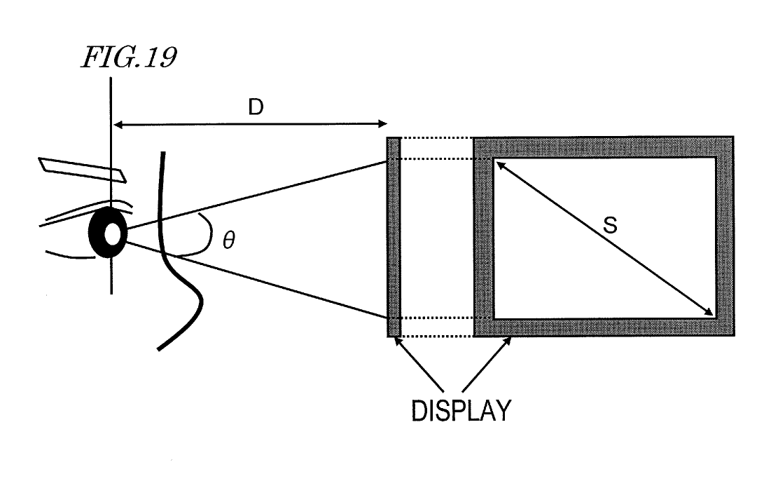

Herein, D is a distance between the frontmost portion of an eyeball (hereinafter referred to as the “eye position”) of a participant and a display; and S is the diagonal length of an object which is defined on the display (e.g., a region in which a video is presented). FIG. 19 schematically shows an example of determining the diagonal length (S) of an object for viewing angle calculation.

The auditory event-related potential measurement system according to the present disclosure reduces changes in the arousal level of a user by presenting a video in a size which is considered appropriate, in addition to auditory stimulations. Then, an auditory event-related potential which is much less affected by changes in arousal level, and an electrical noise occurring due to an eye movement during video watching is measured.

The outline of one implementation of the present invention is as follows.

An auditory event-related potential measurement system as one implementation of the present invention includes: a size determination section configured to determine a size of a region within a video to be presented to a user so that the region has a viewing angle between diagonal corners in a range greater than 2 degrees and smaller than 14 degrees; a video output section configured to present to the user a video including a region of the size determined by the size determination section; an auditory stimulation output section configured to present an auditory stimulation to the user during a period in which the video is being presented to the user; a biological signal measurement section configured to measure an electroencephalogram signal of the user; and an electroencephalogram processing section configured to acquire an event-related potential from the electroencephalogram signal as reckoned from a point in time at which the auditory stimulation is presented.

In one embodiment, the auditory event-related potential measurement system further includes a calculation section configured to take an arithmetic mean of the event-related potential acquired by the electroencephalogram processing section.

In one embodiment, the auditory event-related potential measurement system further includes a distance measurement section configured to measure a distance from an eye position of the user to the video output section, wherein the size determination section determines the size of the region within the video based on the distance.

In one embodiment, the distance measurement section measures the distance at a predetermined timing; and based on the measured distance, the size determination section changes the size of the region within the video while the event-related potential is being measured.

In one embodiment, the auditory event-related potential measurement system further includes a video reproduction processing section configured to retain at least one type of video content to be presented to the user, and configured to perform a reproduction process of a retained video content.

In one embodiment, the video content does not contain audio information.

In one embodiment, when the video content contains any audio information, the video output section prohibits outputting of the audio.

In one embodiment, the video reproduction processing section retains a plurality of types of video contents; and the video reproduction processing section performs a reproduction process of a video content selected by the user from among the plurality of types of video contents.

In one embodiment, the auditory event-related potential measurement system further includes an auditory stimulation generation section configured to determine which of right and left ears of the user the auditory stimulation is to be presented to, configured to determine a frequency and a sound pressure of the auditory stimulation, and configured to generate the auditory stimulation with characteristics so determined.

In one embodiment, the size determination section determines the size of the video so that a viewing angle between diagonal corners of the entire video presented to the user is in a range greater than 2 degrees and smaller than 14 degrees.

In one embodiment, the size determination section determines the size of a partial region within the video so that a viewing angle between diagonal corners of the partial region within the video presented to the user is in a range greater than 2 degrees and smaller than 14 degrees.

An auditory event-related potential measurement method as one implementation of the present invention includes: determining a size of a region within a video to be presented to a user so that the region has a viewing angle between diagonal corners in a range greater than 2 degrees and smaller than 14 degrees; presenting to the user a video including a region of the size determined by the step of determining the size; presenting an auditory stimulation to the user during a period in which the video is being presented to the user; measuring an electroencephalogram signal of the user; and acquiring an event-related potential from the electroencephalogram signal as reckoned from a point in time at which the auditory stimulation is presented.

A computer program as one implementation of the present invention is a computer program stored on a non-transitory computer-readable medium, and to be executed by a computer provided in an auditory event-related potential measurement apparatus of an auditory event-related potential measurement system, the computer program causing the computer to execute: determining a size of a region within a video to be presented to a user so that the region has a viewing angle between diagonal corners in a range greater than 2 degrees and smaller than 14 degrees; presenting to the user a video including a region of the size determined by the step of determining the size; presenting an auditory stimulation to the user during a period in which the video is being presented to the user; acquiring an electroencephalogram signal of the user; and acquiring an event-related potential from the electroencephalogram signal as reckoned from a point in time at which the auditory stimulation is presented.

An auditory event-related potential measurement apparatus as one implementation of the present invention is an auditory event-related potential measurement apparatus for use in an auditory event-related potential measurement system having a video output section, an auditory stimulation output section, and a biological signal measurement section, including: a size determination section configured to determine a size of a region within a video to be presented to a user so that the region has a viewing angle between diagonal corners in a range greater than 2 degrees and smaller than 14 degrees; and an electroencephalogram processing section configured to acquire an event-related potential from an electroencephalogram signal measured by the biological signal measurement section. When the auditory stimulation output section presents an auditory stimulation to the user during a period in which the video output section is presenting to the user a video including a region of the size determined by the size determination section, the electroencephalogram processing section acquires an event-related potential from the electroencephalogram signal as reckoned from a point in time at which the auditory stimulation is presented.

With an auditory event-related potential measurement system according to the present disclosure, during an auditory event-related potential measurement, a video is presented in a size which is considered appropriate is presented in addition to auditory stimulations, thus reducing fluctuations in the auditory event-related potential due to changes in the arousal level of the user, and realizing a highly accurate auditory event-related potential measurement. In particular, it is effective for the measurement of auditory event-related potential in response to auditory stimulations at sound pressures lower than a sound pressure which is generally evaluated to be the UCL. As a result, the accuracy of user hearing evaluation is improved, thus realizing a hearing aid adjustment which does not leave much to be desired by the user, for example.

Hereinafter, the background and findings which led to the present disclosure will be described. Thereafter, the auditory event-related potential measurement system will be described as embodiments, and the construction and operation of the auditory event-related potential measurement apparatus will be described in detail.

(Background of the Present Disclosure)

As mentioned earlier, in any auditory event-related potential measurement where monotonous auditory stimulations at sound pressures lower than a sound pressure which is generally evaluated to be the UCL are repeated, the user may not be able to maintain his or her arousal level. This causes changes in the auditory event-related potential waveform that are associated with arousal level fluctuations.

In order to suppress arousal level fluctuations of the user, the inventors have paid attention to a method of presenting visual stimulations (video) during an auditory event-related potential measurement; visual stimulations are of a different modality from that of auditory stimulations. Specifically, the inventors have given thought to a method which, while simultaneously presenting auditory stimulations and visual stimulations (video), measures auditory event-related potentials that are evoked by the auditory stimulations. Examples of videos which can suppress arousal level fluctuations include movies, TV programs such as dramas or sport broadcasting, and so on. However, during such video watching, an eye-movement related electrooculogram occurs, and is mixed in the electroencephalogram to become a noise with a large amplitude (which in the present specification is referred to as an “electrooculographic noise”). Therefore, a video presentation method needs to be devised which suppresses arousal level fluctuations and which is not susceptible to the influence of an electrooculogram. The inventors have realized an auditory event-related potential measurement which avoids the influences of arousal level fluctuations and an electrooculogram by presenting a video in an appropriately selected size.

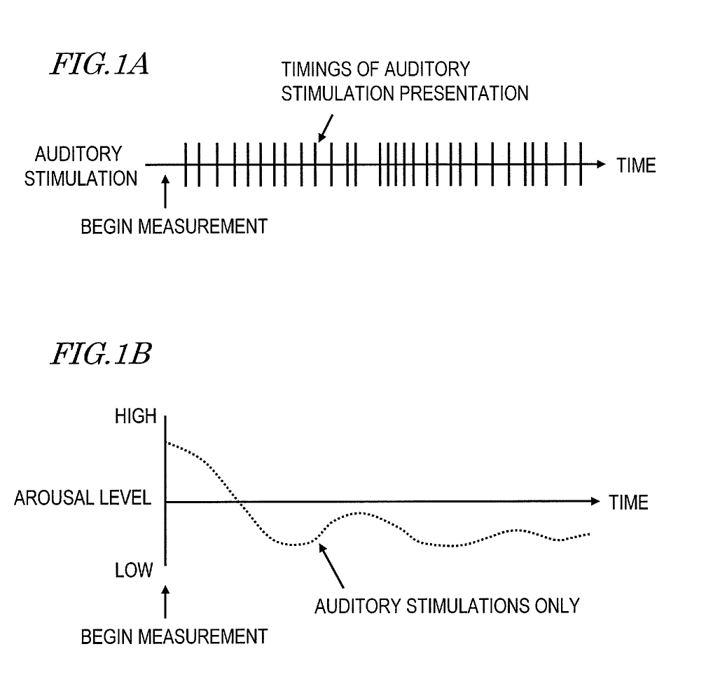

FIG. 1A shows an experimental paradigm of a conventional auditory event-related potential measurement. The horizontal axis represents time, against which timings of auditory stimulations are schematically indicated by vertical lines. In order to reduce noises such as the background electroencephalogram through arithmetic mean, auditory stimulations are repetitively presented. For example, assuming that each auditory stimulation has a duration of 100 ms, the stimulation intervals have a mean value of 1 second, and the number of repetitions is 30 times, then, about 30 seconds of time is required for an auditory event-related potential measurement with respect to one frequency, one sound pressure, and one ear.

Therefore, in the case of measuring auditory event-related potentials for five frequencies, five sound pressures, and both ears in order to make a hearing evaluation of a user, for example, a simple calculation would indicate that about 25 minutes (30×5×5×2 seconds) is required. Thus, the user needs to keep hearing monotonous auditory stimulations for a total of about 25 minutes, which makes it difficult to maintain his or her arousal level especially in the case of receiving auditory stimulations at sound pressures lower than a sound pressure which is generally evaluated to be the UCL. FIG. 1B shows imaginary arousal level fluctuations of a user during the auditory event-related potential measurement. The horizontal axis represents time, whereas the vertical axis represents the arousal level. FIG. 1B illustrates an imaginary manner in which the arousal level may lower with lapse of time since the beginning of an auditory event-related potential measurement.

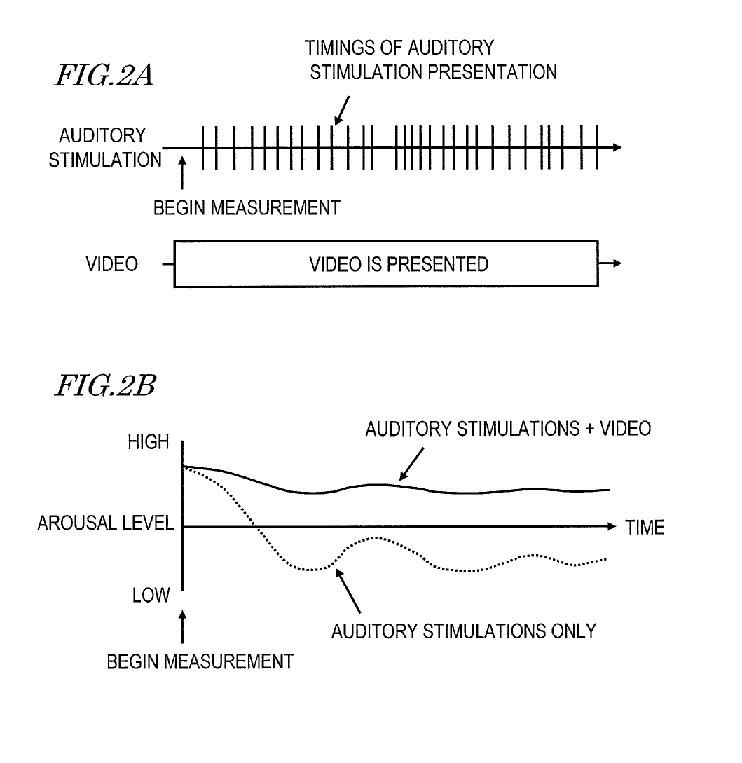

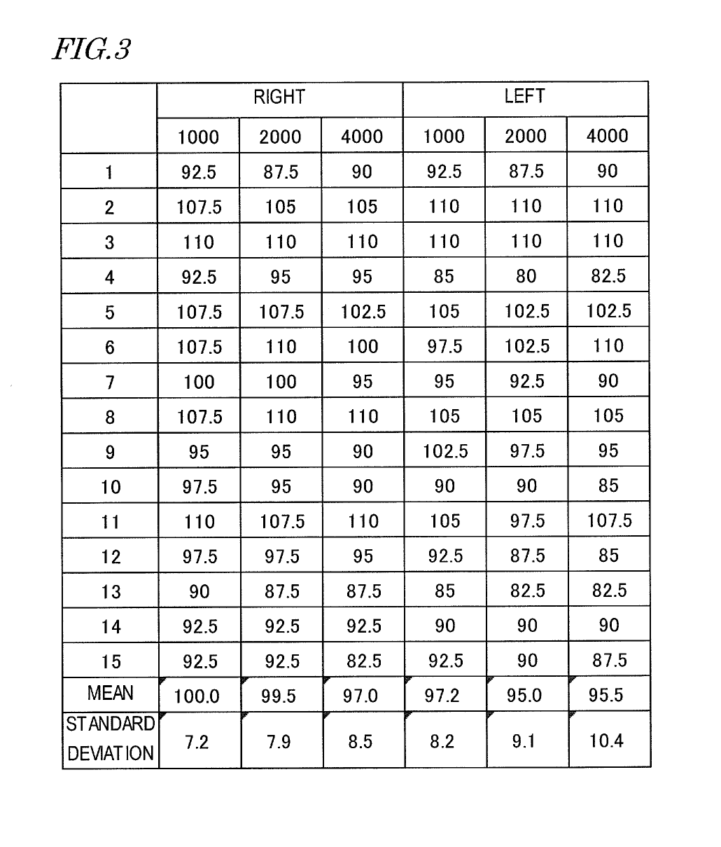

FIG. 2A shows an auditory event-related potential measurement paradigm where a video is concurrently presented. The inventors have paid attention to an method of auditory event-related potential measurement shown in FIG. 2A. In order to suppress a decrease in the arousal level of the user during the auditory event-related potential measurement, auditory stimulations are presented while presenting a video. FIG. 2B shows imaginary arousal level fluctuations of a user during the auditory event-related potential measurement in a similar manner to FIG. 1B. It is considered that, due to the video presentation, decrease in the arousal level of the user is suppressed, so that the arousal level is maintained relatively high.

Hereinafter, a UCL estimation method based on an index which is an event-related potential in response to an auditory stimulation at a sound pressure lower than a sound pressure which is generally evaluated to be the UCL will be described first; this method was found through experiments conducted by the inventors. Then there will be described a highly accurate auditory event-related potential measurement method that suppresses arousal level fluctuations through simultaneous presentation of a video, which has been devised by the inventors in view of the above problems.

(Experiments for UCL Estimation Based on Event-Related Potentials in Response to Auditory Stimulations that are not Loud)

1-1. Experimental Outline

The inventors have conducted the following two experiments in order to collect fundamental data for making an uncomfortable sound pressure estimation based on an index which is an auditory event-related potential in response to a pure tone at a sound pressure lower than a sound pressure which is generally evaluated to be the UCL.

One is a subjective report experiment of measuring a UCL based on subjective reporting. The subjective report experiment was conducted before and after an electroencephalogram measurement experiment (see below). The UCL data obtained from this subjective report experiment was used as reference data against which any brain-based estimation was to be contrasted.

Another is an electroencephalogram measurement experiment of measuring responses to auditory stimulations. In the electroencephalogram measurement experiment, pure tones of the same frequency were presented totaling three times in succession, with monotonously-descending sound pressure changes of every 5 dBHL, and event-related potentials in response to the respective auditory stimulations of first to third sounds were measured. Hereinafter, auditory stimulations being presented a plurality of times successively with monotonously-descending sound pressure changes may also be referred to as “decrescendo stimulations”. Event-related potentials to such auditory stimulations were acquired for use as data in UCL value estimation.

As a result, the inventors have found that a UCL conforming to subjective reporting can be estimated even when decrescendo stimulations are presented at sound pressures lower than a sound pressure which is generally evaluated to be the UCL, by applying linear discrimination to a change pattern of wavelet coefficients calculated through wavelet transform of event-related potentials in response to the first to third sounds.

Herein, it is assumed that a sound pressure lower than a sound pressure which is generally evaluated to be the UCL varies depending on the HTL value. For example, according to works of Pascoe (Pascoe, D. P. (1988). (Clinical measurements of the auditory dynamic range and their relation to formulas for hearing aid gain. In lensen. H. l. (Ed.) Hearing Aid Fitting: Theoretical and Practical Views 13th Danavox Symposium. Copenhagen: Stougaard.)), a value which is at least 5 dB lower than an estimated UCL value for each HTL value may be designated the aforementioned “sound pressure lower than a sound pressure which is generally evaluated to be the UCL”. Note that it is when an auditory stimulation has a sound pressure which is higher than the HTL that any event-related potential will be induced in response to that auditory stimulation. In other words, a range of sound pressures lower than a sound pressure which is generally evaluated to be the UCL should be a range of sound pressures higher than the HTL. With this technique, a UCL estimation is achieved in a short time and with a high accuracy, without presenting overbearing sounds.

Hereinafter, the experiments conducted by the inventors and the results thereof, and characteristic features of electroencephalograms which have been found through the inventors' analysis will be described in detail. Thereafter, as an embodiment of the present disclosure, an outline of the auditory event-related potential measurement system, detailed configuration thereof, and its operation will be described.

(Experimental Conditions)

1-2. UCL Subjective Report Experiment and Electroencephalogram Measurement Experiment

1-2-1. UCL Subjective Report Experiment

The experimental participants were 15 adults, who were no longer in school, having normal hearing (28 to 49 years old).

The subjective report experiment was conducted before and after the electroencephalogram measurement experiment (see below). Similarly to Non-Patent Document 1, discontinuous sounds were presented by ascending method using an audiometer, and an uncomfortably loud sound pressure was reported by each experimental participant, this sound pressure being defined as the UCL. For each of three frequencies (1000, 2000, 4000 Hz) to be presented in the electroencephalogram measurement experiment, the inventors took measurement for both ears, one ear at a time. In order to prevent the experimental participants from anticipating the sound pressure, the sound pressure at the start of the experiment was randomly selected from among 60, 65, and 70 dB. The sound pressure of the discontinuous sounds ascended by every 5 dB. An uncomfortably loud sound pressure was reported by raising a hand. Immediately after the participant raised a hand, the sound presentation was stopped, and the sound pressure was recorded as a subjective UCL value.

Hereinafter, results of the subjective report experiment will be described.

All participants were people with normal hearing. However, the results of the subjective report experiment greatly differed from individual to individual. For example, for the same frequency, there was a difference of 40 dB at the most. This indicates that the definition of “uncomfortably loud” may greatly from individual to individual. Thus, it can be said that UCL measurement through subjective reporting is difficult.

FIG. 3 shows UCL measurement results of individuals which were measured through subjective reporting in the subjective report experiment. FIG. 3 indicates average values of two measurement results each. The sound pressure is in units of dBHL. As can be seen from the standard deviation for the right or left ear and for each different frequency shown in FIG. 3, there are some fluctuations in the subjective UCL value. It can be seen that there are large fluctuations among individuals.

1-2-2. Electroencephalogram Measurement Experiment

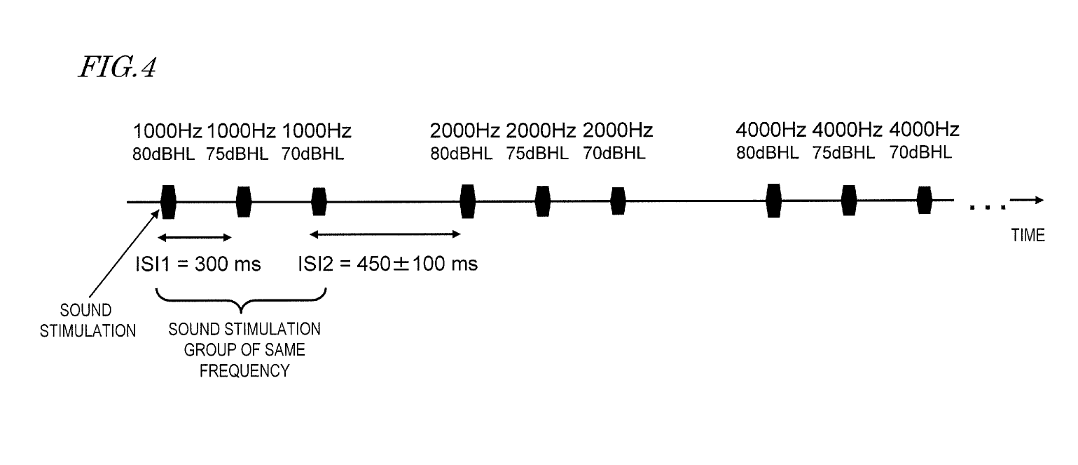

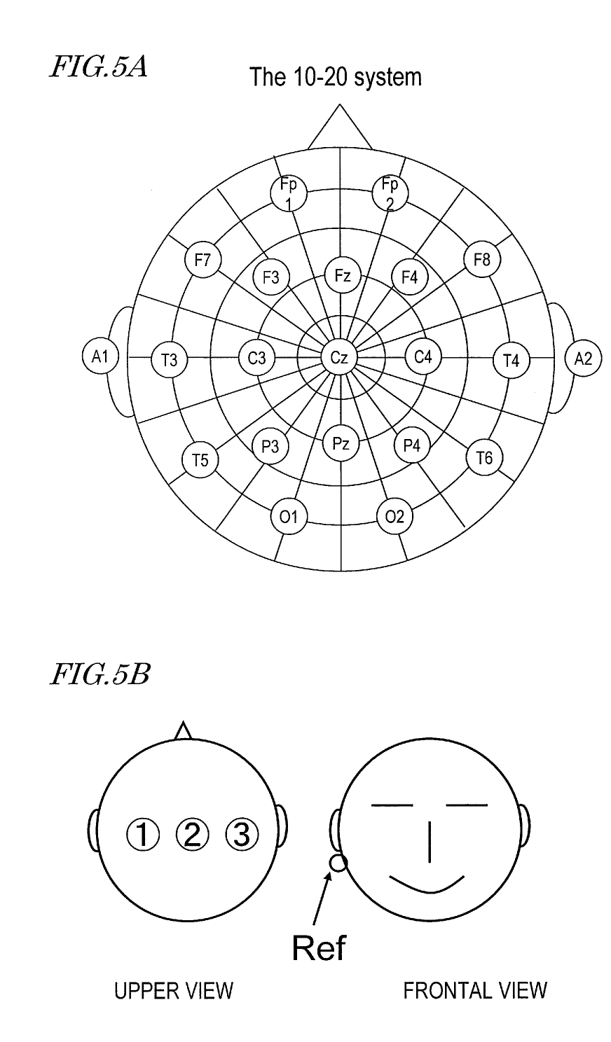

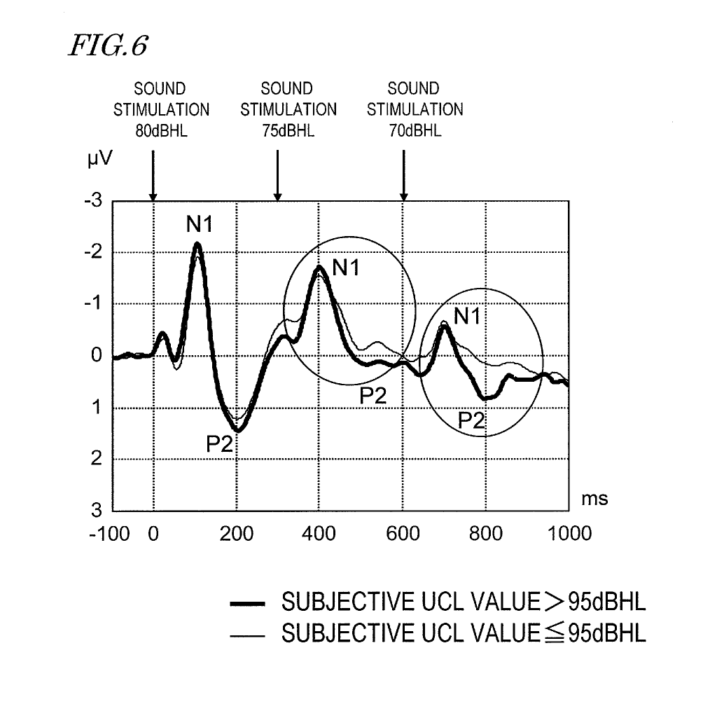

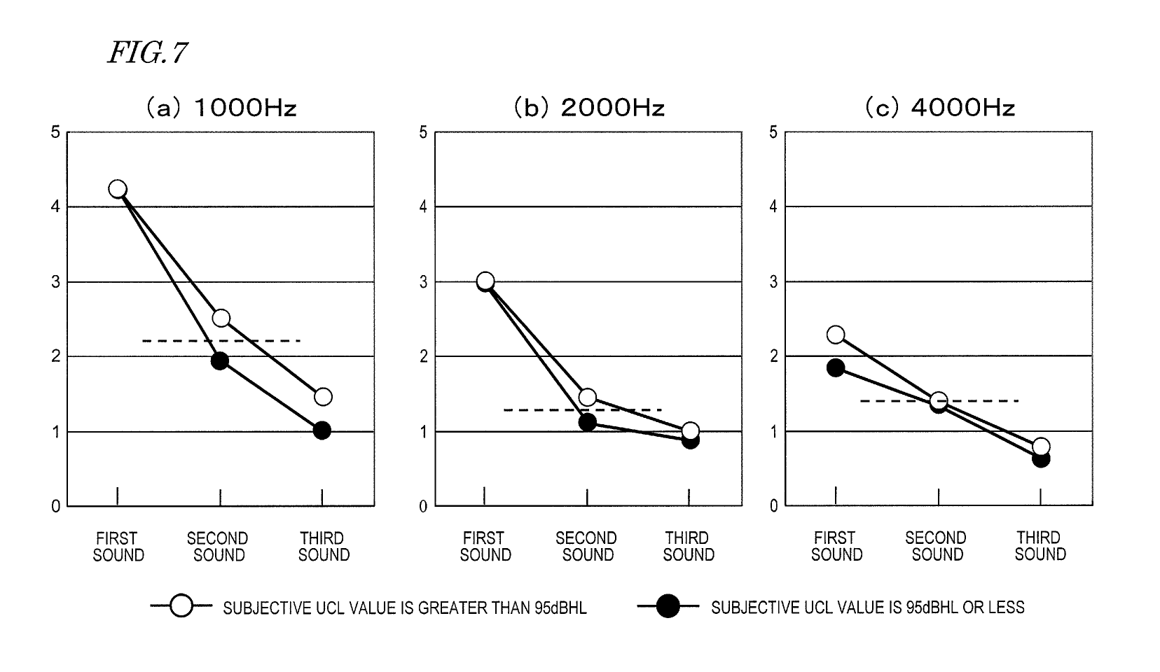

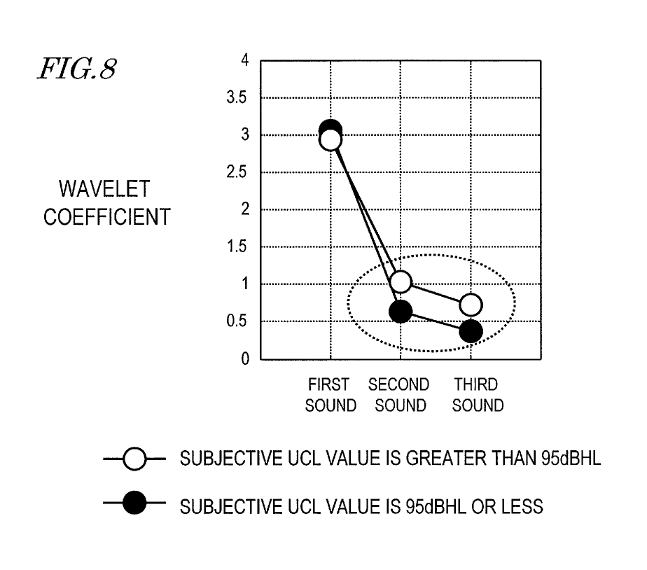

In the electroencephalographic experiment, for each of three frequencies (1000 Hz, 2000 Hz, 4000 Hz), auditory stimulations were presented at three sound pressures (80, 75, 70 dBHL) lower than a sound pressure which is generally evaluated to be the UCL. The three sound pressures were monotonously descending. Then, a characteristic change in the event-related potential for each auditory stimulation was examined. Hereinafter, with reference to FIG. 4, FIGS. 5A and 5B, and FIG. 6, the experimental setting and experimental results of the electroencephalogram measurement experiment will be described.

The experimental participants were the same 15 adults in the subjective report experiment, who were no longer in school (28 to 49 years old) and who had normal hearing.

As the auditory stimulations, the inventors used toneburst sounds with a duration of 50 ms. Each auditory stimulation had a rise (rise) and (fall) of 3 ms each. For each of the three frequencies (1000, 2000, 4000 Hz) and for each of the right or left ear, characteristic amount variation in the event-related potential against changing sound pressure was examined, by using auditory stimulations of the three sound pressures (80, 75, 70 dBHL). A group of auditory stimulations pertaining to the same frequency will be referred to as an “auditory stimulation group”.

The auditory stimulations contained in the auditory stimulation group were with respect to the same ear at predetermined intervals. Each auditory stimulation was presented to one ear through headphones.

FIG. 4 schematically shows auditory stimulations presented in the electroencephalogram measurement experiment.

The participants were instructed that there was no need to pay attention to the auditory stimulations. The interval between auditory stimulations within an auditory stimulation group of the same frequency (ISI1 in FIG. 4) was fixed at 300 ms. Moreover, the interval between auditory stimulation groups (ISI2 in FIG. 4) was randomly decided within a range of 450±100 ms. The auditory stimulation group for the right or left ear and for: each different frequency was repeated 30 times (totaling in 180 auditory stimulation groups).

In order to reduce taming (habituation) of the auditory evoked potential due to successive presentation of the same auditory stimulation group, the inventors determined the frequency and the ear for which to present the auditory stimulation group under the following constraints.

the frequency is selected to be different from that of an immediately previous auditory stimulation group.

the ear to which the auditory stimulation group is presented is randomly selected between right or left. However, in order to ensure randomness of stimulations between the right and left ears, not more than four auditory stimulation groups are successively presented to either the right or left ear.

Next, the positions of electrodes to be worn for electroencephalogram measurement will be described. FIG. 5A shows electrode positions according to the International 10-20 system (10-20 System). FIG. 5B shows the positions of electrodes worn in this experiment. In FIG. 5B, circled numbers 1, 2, and 3 represent electrode positions C3, Cz, and C4, respectively. The inventors recorded the electroencephalogram from C3, Cz, and C4 (the International 10-20 system) on the scalp, on the basis of the right mastoid. A “mastoid” is a protrusion of the cranium below the hind root of an ear. FIG. 5B shows the mastoid position as “Ref”.

The electroencephalogram was measured with a sampling frequency of 1000 Hz and a time constant of 0.3 seconds, by applying an analog low-pass filter at 30 Hz. The entire time slot of electroencephalogram data measured was subjected to a 5-20 Hz digital band-pass filter off-line. Thereafter, as an event-related potential in response to an auditory stimulation for the right or left ear, for each different frequency, and for each different sound pressure, a waveform from −100 ms to 400 ms was cut out based on the respective auditory stimulation as a starting point. As used herein, “−100 ms” means a point in time which is 100 milliseconds before the point in time at which an auditory stimulation is presented.

Moreover, for each auditory stimulation, an electroencephalogram waveform in a range from 0 ms to 300 ms of the event-related potential was subjected to a continuous wavelet transform to derive a wavelet coefficient with respect to time and frequency. As a mother wavelet, the Mexican hat function (φ(t)=(1t̂2)exp(t̂2/2)) was used.

The waveforms and wavelet coefficients of event-related potential were arithmetic-meaned, for each individual person, each of the right or left ear, each frequency, and every auditory stimulations of first to third sounds. These will be referred to as, respectively, the arithmetic mean waveform and the arithmetic mean wavelet coefficient. Those trials which exhibited an amplitude in absolute value of 50 μV or more at any electrode were excluded from the total arithmetic mean and arithmetic mean, because they presumably are under the influence of noises, e.g., eye movements and blinks.

Then, as a characteristic amount of the event-related potential potentially serving as an index of uncomfortable sound pressure, average values of the arithmetic mean wavelet coefficients over a frequency range from 5 Hz to 12.5 Hz were calculated in every time range of 50 ms (hereinafter referred to as wavelet characteristic amounts).

1-3. Results

Hereinafter, results of the electroencephalogram measurement experiment will be described.

First, in order to confirm that an index of uncomfortable sound pressure estimation exists in the event-related potential against changing sound pressure, arithmetic-meaned event-related potentials were compared on the basis of the subjective UCL value. In order to estimate an uncomfortable sound pressure based on event-related potential, a difference in event-related potential needs to exist that reflects a subjective UCL value of each participant. Now, as discussed above, the subjective UCL value can only be an index that is prone to fluctuations among participants, because of different personalities existing with respect to overbearing sounds. This makes it difficult to identify the presence or absence of a characteristic amount that reflects a subjective UCL value from the data of each individual person. Therefore, in order to reduce such fluctuations, event-related potentials were arithmetic-meaned and compared while making a distinction between large subjective UCL values and small subjective UCL values. Specifically, an arithmetic mean was taken with respect to the cases where the subjective UCL value for each participant and for each frequency was greater than 95 dBHL, or the cases where it was equal to or less than 95 dBHL, and these results were compared. Note that 95 dBHL is a value near the center of the subjective UCL values of all participants obtained from the subjective report experiment, and there were substantially the same number of cases where the subjective UCL value was greater than 95 dBHL as the cases where it was equal to or less than 95 dBHL.

FIG. 6 shows total arithmetic mean electroencephalogram waveforms for different subjective UCL values. Each electroencephalogram waveform subjected to the total arithmetic mean was measured at the central portion (Cz), from 100 ms before the first sound in the auditory stimulation group until 400 ms after the third sound. A thick line indicates the case where the subjective UCL value is greater than 95 dBHL, whereas a thin line indicates the case where the subjective UCL value is 95 dBHL or less. The horizontal axis represents time in units of ms, and the vertical axis represents potential in units of μV. On the horizontal axis, 0 ms denotes a point at which the first sound is presented. It can be seen that, as reckoned from each timing of auditory stimulation presentation (indicated by an arrow), a negative N1 component is induced at about 100 ms and a positive P2 component is induced at about 200 ms. It can also be seen that there is a difference in the event-related potential at the second sound presentation and thereafter, depending on whether the subjective UCL value is high or low. Specifically, the N1-P2 amplitude is larger in the case where the subjective UCL value is greater than 95 dBHL (indicated by the thick line), than in the case where the subjective UCL value is 95 dBHL or less. This suggests an ability to estimate a UCL based on an index which is the difference in event-related potential at the second sound and thereafter. Note that an “N1-P2 amplitude” represents the absolute value of a difference between the negative amplitude of an N1 component and the positive amplitude of a P2 component.

Portions (a) to (c) of FIG. 7 show a relationship between greater or smaller subjective UCL values and the N1-P2 amplitude. For each different frequency, portions (a) to (c) of FIG. 7 show N1-P2 amplitude in response to the first to third sounds, with respect to the case where the subjective UCL value is greater than 95 dBHL and the case where the subjective UCL value is 95 dBHL or less. The N1-P2 amplitude is defined as the absolute value of a difference between an N1 amplitude and a P2 amplitude. The N1 amplitude is a zone average potential from 90 ms to 110 ms after the presentation of each auditory stimulation of the first to third sounds. Similarly, the P2 amplitude is a zone average potential from 190 ms to 210 ms after each auditory stimulation presentation. In the case where the subjective UCL value is greater than 95 dBHL, the N1-P2 amplitude in response to the first to third sounds is 4.24 μV, 2.51 μV, 1.45 μV at 1000 Hz; 2.99 μV, 1.45 μV, 1.00 μV at 2000 Hz; and 2.28 μV, 1.40 μV, 0.78 μV at 4000 Hz.

In the case where the subjective UCL value is 95 dBHL or less, the N1-P2 amplitude in response to the first to third sounds is 4.24 μV, 1.95 μV, 0.99 μV at 1000 Hz; 2.95 μV, 1.11 μV, 0.88 μV at 2000 Hz; and 1.84 μV, 1.33 μV, 0.63 μV at 4000 Hz. At any frequency, the N1-P2 amplitude in response to the second and third sounds is larger in the case where the subjective UCL value is greater than 95 dBHL than in the case where the subjective UCL value is 95 dBHL or less. This indicates that, depending on the subjective UCL value, the event-related potential for changing sound pressure varies at least in terms of N1-P2 amplitude.

Next, the inventors examined the relationship between the subjective UCL value and the wavelet characteristic amount. Then, the inventors conducted a discriminant analysis in order to ascertain the accuracy of an uncomfortable sound pressure estimation using changes in this characteristic amount.

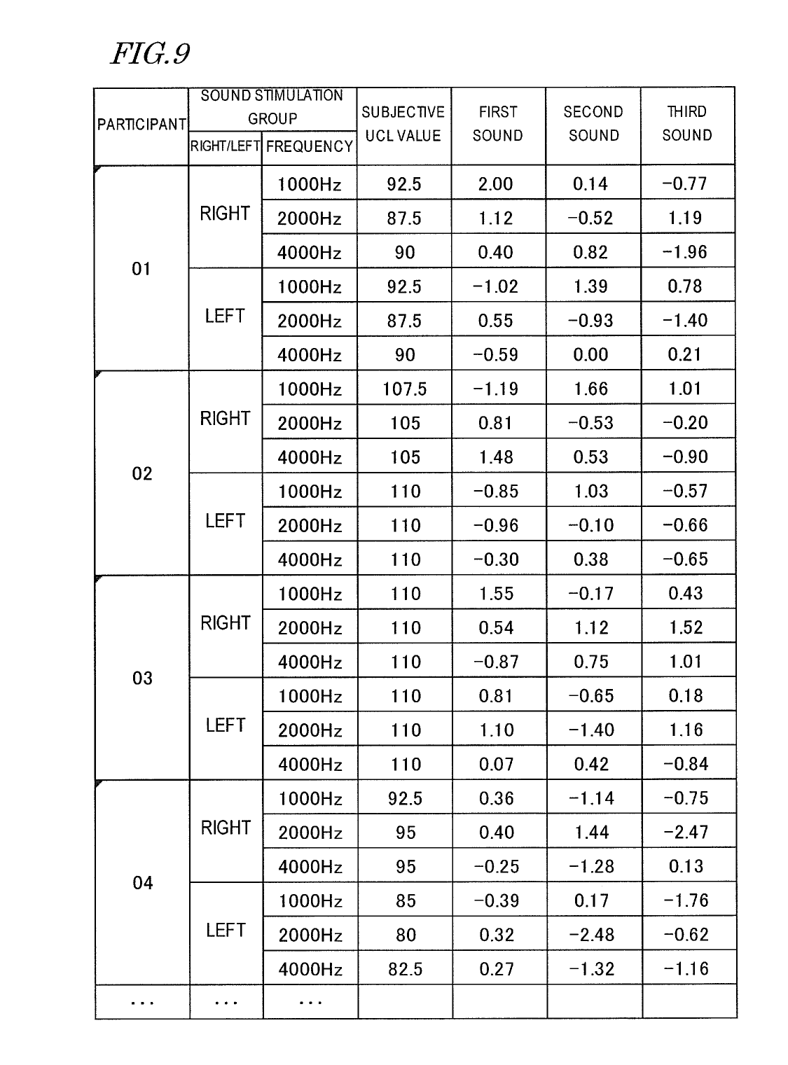

FIG. 8 shows wavelet characteristic amounts in response to the first to third sounds, under different conditions and different subjective UCL values. As exemplary results, FIG. 8 indicates wavelet characteristic amounts in a time slot from 201 ms to 250 ms, this time slot defining a time zone as reckoned from a point at which each auditory stimulation is presented. It can be seen that, although the difference in wavelet characteristic amount is small with respect to the first sound (80 dBHL), the wavelet characteristic amounts in response to the second sound (75 dBHL) and the third sound (70 dBHL) differ depending on the subjective UCL value. Specifically, the wavelet characteristic amount in response to the second and third sounds are larger in the case where the subjective UCL value is greater than 95 dBHL, than in the case where the subjective UCL value is 95 dBHL or less. This indicates that, depending on the subjective UCL value, the event-related potential for changing sound pressure varies in terms of wavelet characteristic amount.

In order to ascertain the accuracy of an uncomfortable sound pressure estimation using characteristic amount variation in the event-related potential, the inventors have conducted a discriminant analysis. Linear discrimination was used as the technique of discriminant analysis, which was conducted by allowing the subjective UCL value for each of the right or left ear and for each frequency obtained through the aforementioned subjective report experiment to be “trained” with a wavelet characteristic amount of an event-related potential for each sound pressure. In order to find characteristic amounts that are suitable for UCL estimation, the error of each characteristic amount (alone or in combination with any other(s)) with respect to the subjective UCL value was ascertained, and a comparison was made between errors resulting from different numbers of characteristic amounts used in combination.

Hereinafter, the data to be used in linear discrimination, and the linear discrimination conducted will be described. FIG. 9 shows an example of data used in an uncomfortable sound pressure estimation. Each subjective UCL value shown in FIG. 9 was measured through the subjective report experiment for each participant, each of the right or left ear, and each frequency. In FIG. 9, the columns corresponding to the first to third sounds show wavelet characteristic amounts (at 201 ms to 250 ms after auditory stimulation) of the event-related potentials in response to the first to third sounds of an auditory stimulation group. These characteristic amounts for each auditory stimulation group were associated with the respective subjective UCL value, for use as training data in a linear discrimination to be conducted.

The inventors conducted the linear discrimination by using target data against training data. The target data for linear discrimination was the characteristic amounts of the event-related potentials for the auditory stimulation group, taken for a given participant. The training data was generated from the characteristic amounts of event-related potentials of other people. Moreover, the inventors generated the training data from the characteristic amounts of the event-related potentials of other people for each condition, each of the right or left ear, and each frequency.

For example, if the target data for linear discrimination was that of participant 01 for the right ear and 1000 Hz, the training data was generated from the characteristic amounts of the data of the event-related potential for the right ear and 1000 Hz from a participant other than participant 01. As the characteristic amounts, the aforementioned wavelet characteristic amounts (time range 50 ms) were used. In order to explore the possibility of uncomfortableness sound pressure estimation, in the case where a plurality of characteristic amounts were to be employed in combination, characteristic amounts were added in extra columns, in either the target data for linear discrimination or the training data. For example, if wavelet characteristic amounts from 151 ms to 200 ms and wavelet characteristic amounts from 201 ms to 250 ms were to be employed in combination, in addition to the first to third columns being allocated to the characteristic amounts in response to the first to third sounds regarding the former, fourth to sixth columns were allocated to the characteristic amounts in response to the first to third sounds regarding the latter. An “estimation error” was defined as the absolute value of a difference between a subjective UCL value and a result of uncomfortable sound pressure estimation. Accuracy of estimation was measured on the basis of an average estimation error, which was obtained by averaging the estimation errors of all participants with respect to right and left and all frequencies.

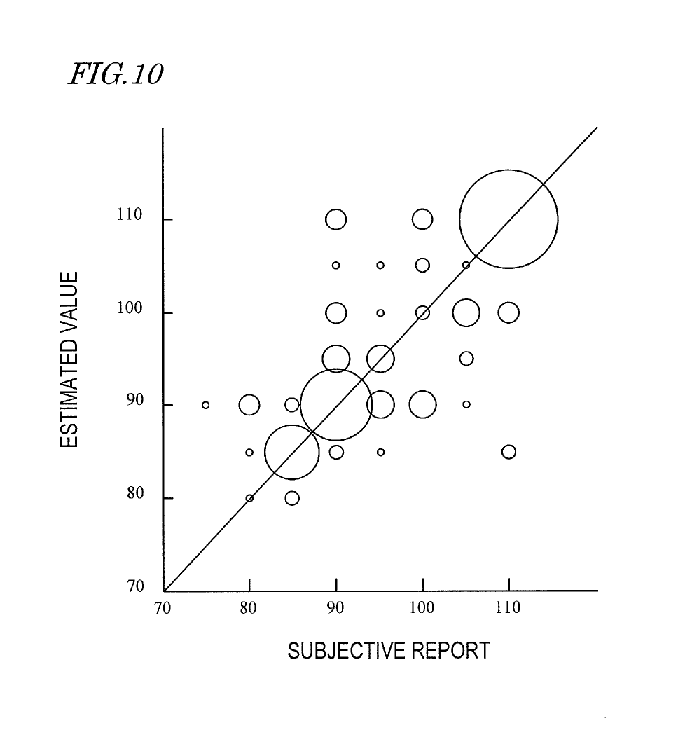

FIG. 10 shows, as exemplification of linear discrimination results, distributions under different conditions of results of uncomfortable sound pressure estimation based on subjective UCL values and linear discrimination, in the case where five characteristic amounts are used in combination. The analysis was conducted for each condition, each of the right or left ear, and each frequency; however, FIG. 10 shows the results altogether, irrespective of the right or left ear or frequency. As indicated by the scale in FIG. 10, the horizontal axis represents subjective UCL values in units of dBHL, and the vertical axis represents uncomfortable sound pressure estimation values in units of dBHL. Results of uncomfortable sound pressure estimation with respect to subjective UCL values are indicated by ◯ symbols as lattice points. The size of any ◯ symbol reflects the frequency distribution of the particular estimation result. The average estimation error was 5.2 dB. From these results, it can be seen that uncomfortable sound pressures which are correlated with the subjective UCL values have successfully been estimated, although there are some fluctuations.

Note that, without being limited to wavelet characteristic amounts, P1-N1 amplitude or N1-P2 amplitude information may be utilized in making a discriminant analysis.

Note that training data may be generated irrespective of the right or left ear and irrespective of sound frequency.

In the present specification, in order to define a component of an event-related potential, a point in time after the lapse of a predetermined time since a given point is expressed by referring to a “latency of about 100 ms”, for example. This means possible inclusion of a range around the specific point of 100 ms. Generally speaking, there are 30 to 50 ms of differences (shifts) in event-related potential waveform between individuals, according to table 1 on p. 30 of “JISHOUKANRENDENI (ERP) MANYUARU—P300 WO CHUSHINNI—(or “Event-Related Potential (ERP) Manual—mainly concerning P300—”), edited by Kimitaka KAGA et al., Shinohara Shuppan Shinsha, 1995)“. Therefore, the terms “about X ms” and “near X ms” mean that a breadth of 30 to 50 ms may exist before or after X ms (e.g., 100 ms±30 ms, 200 ms±50 ms).

Thus, it has been made clear through the subjective report experiment and electroencephalogram measurement experiment conduced by the inventors that, when pure tones of the same frequency are presented totaling three times in succession at monotonously-descending sound pressure changes within a range of sound pressures lower than a sound pressure which is generally evaluated to be the UCL, it is possible to estimate an uncomfortable sound pressure by using characteristic amounts concerning the wavelet coefficients of electroencephalograms in response to the respective auditory stimulations of first to third sounds.

(Experiment of Identifying a Presumably Appropriate Video Size)

In view of the aforementioned problems of arousal level fluctuations during the auditory event-related potential measurement, the inventors have conducted an auditory event-related potential measurement experiment for the purposes of: (1) confirming that arousal level fluctuations during the auditory event-related potential measurement are suppressed by simultaneously presenting a video; and (2) identifying the video size which is considered appropriate for presentation during the auditory event-related potential measurement. As a result, the inventors have (1) confirmed that arousal level fluctuations of a user are suppressed by simultaneously presenting a video, and (2) found that the presumably appropriate the video size is defined by a viewing angle between diagonal corners in the video being greater than 2 degrees and smaller than 14 degrees. This will be described in detail below.

One commonly-used method for reducing the influence of electrooculographic noise is to provide an electrode for monitoring an electrooculogram around the eyeball, multiply an electrooculogram which is measured at that electrode by a transfer factor of 1 or less, and subtract the product from an electroencephalogram which is measured on the head. However this has a problem in that an electrode needs to be worn around the eyeball, which is cumbersome to the user. Therefore, as a prerequisite, the present specification assumes an auditory event-related potential measurement which is made without providing any electrode for electrooculogram monitoring.

Since the frequency of electrooculographic noise is about 10 Hz, its influence can be reduced through frequency filtering if the electroencephalogram signal for measurement has a significantly different frequency. However, the frequency of an auditory event-related potential is about 10 Hz, which is close to that of the electrooculographic noise, thus making it difficult to reduce electrooculographic noise through frequency filtering.

2-1. Experimental Outline

For the aforementioned purpose (1), an auditory event-related potential was measured under a condition (no-video condition) of presenting an auditory stimulation while presenting-a fixation point on a screen, and a condition of presenting an auditory stimulation while presenting a video (video-presented condition). Under the video-presented condition, for the aforementioned purpose (2), videos were presented whose viewing angle between diagonal corners ranged from 2 degrees to 18 degrees (totaling 5 types), which will be respectively referred to as the video-at-2 degrees condition, the video-at-6 degrees condition, the video-at-10 degrees condition, the video-at-14 degrees condition, and the video-at-18 degrees condition. After measurement under each condition, a subjective report concerning the arousal level and eye fatigue was made. Separately, an uncomfortable sound pressure for each frequency was measured through subjective reporting (referred to as the subjective UCL value). Then, based on an error between an uncomfortable sound pressure which is estimated by applying linear discrimination to the auditory event-related potential measured under each condition (referred to as the estimated uncomfortable sound pressure) and the subjective UCL value, the respective conditions of auditory event-related potential measurement were evaluated.

2-2. Method

The experimental participants were 5 adults, who were no longer in school, having normal hearing (32 to 47 years old).

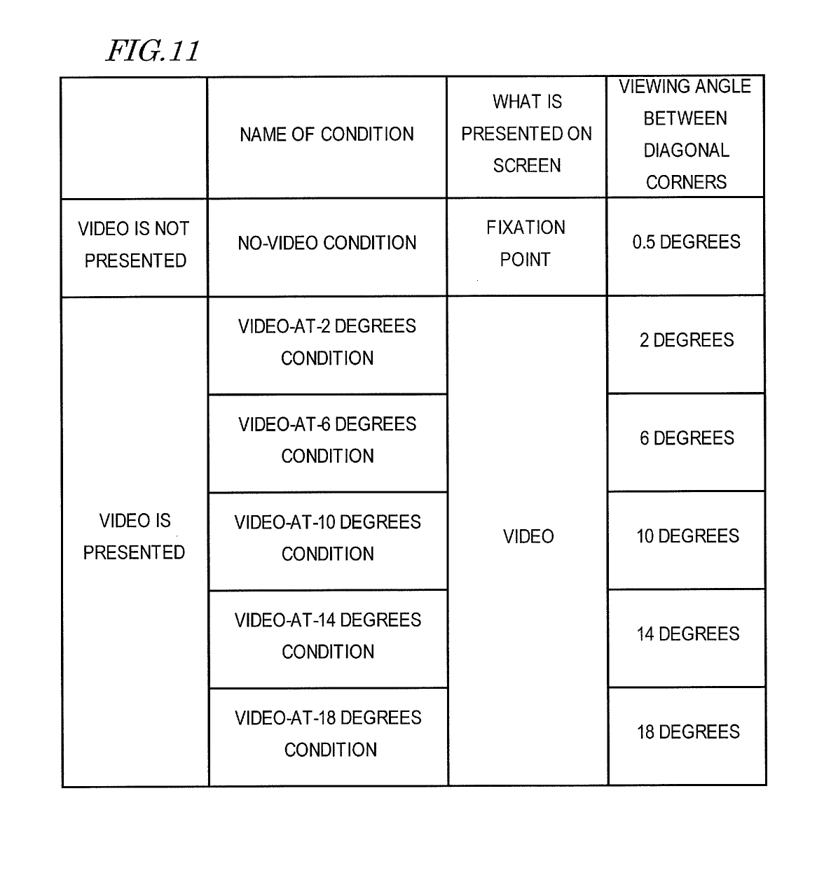

FIG. 11 shows conditions of screen presentation in the auditory event-related potential measurement experiment conducted. The fixation point and the video, if any, were presented on a display which was placed 1 m in front of each participant. The fixation point under the no-video condition was a mouse pointer (arrow) spanning a viewing angle of 0.5 degrees. As for the video under each video-presented condition, a video having a viewing angle as indicated by the numerical contained in its condition name was presented. The condition-to-condition experimental order was counterbalanced between participants. The participants were instructed to stare at the fixation point under the no-video condition, or the video under any of the five video-presented conditions.

The auditory stimulations were the same irrespective of the condition (identical to those in the electroencephalogram measurement experiment described in 1-2-2; FIG. 4). As the auditory stimulations, for each of three frequencies (1000 Hz, 2000 Hz, 4000 Hz), pure tones (rise-fall: 3 ms) of three sound pressures (80, 75, 70 dB HL) were prepared. Then, pure tones of the same frequency were presented in the order of 80 dBHL, 75 dBHL, 70 dBHL, totaling three times in succession, at an interval of 300 ms. The pure tones of the same frequency being presented a total of three times in succession are called an auditory stimulation group. The auditory stimulation group was presented for each one ear. The auditory stimulation group for the right or left ear and for each frequency was repeated 25 times (totaling in 150 auditory stimulation groups). The interval between auditory stimulation groups was 450±50 ms. In order to reduce taming (habituation) of the auditory evoked potential due to successive presentation of the same auditory stimulation group, the frequency and the ear for which to present the auditory stimulation group were determined under the following constraints: the frequency is selected to be different from that of an immediately previous auditory stimulation group; the ear to which the auditory stimulation group is presented is randomly selected between right or left; however, in order to ensure randomness of stimulations between the right and left ears, not more than four auditory stimulation groups are successively presented to either the right or left ear.

The electroencephalogram was recorded from C3, Cz, C4 on the scalp (the International 10-20 system), on the basis of the right mastoid. A “mastoid” is a protrusion of the cranium below the hind root of an ear. FIG. 5A shows electrode positions according to the International 10-20 system (10-20 System). FIG. 5B shows the positions of electrodes worn in this experiment. In FIG. 5B, circled numbers 1, 2, and 3 represent electrode positions C3, Cz, and C4, respectively.

The electroencephalograph was measured with a sampling frequency of 1000 Hz and a time constant of 0.5 seconds, by applying an analog low-pass filter at 30 Hz. The entire time slot of electroencephalogram data measured was subjected to a 5-20 Hz digital band-pass filter off-line. Thereafter, as an event-related potential in response to an auditory stimulation for the right or left ear, for each different frequency, and for each different sound pressure, a waveform from −100 ms to 400 ms was cut out based on the respective auditory stimulation as a starting point. As used herein, “−100=” means a point in time which is 100 milliseconds before the point in time at which an auditory stimulation is presented.

Moreover, for each auditory stimulation, an electroencephalogram waveform in a range from 0 ms to 300 ms of the event-related potential was subjected to a continuous wavelet transform to derive a wavelet coefficient with respect to time and frequency. As a mother wavelet, the Mexican hat function (φ(t)=(1t̂2)exp(t̂2/2)) was used.

The waveform and wavelet coefficients of event-related potential were arithmetic-meaned, for each condition, each individual person, each of the right or left ear, each frequency, and each auditory stimulation group of first to third sounds. These will be referred to as, respectively, the arithmetic mean waveform and the arithmetic mean wavelet coefficient. Those trials which exhibited an amplitude in absolute value of 50 μV or more at any electrode were excluded from the total arithmetic mean and arithmetic mean, because they presumably are under the influence of noises, e.g., eye movements and blinks. Then, as a characteristic amount of the event-related potential potentially serving as an index of uncomfortable sound pressure, average values of the arithmetic mean wavelet coefficients over a frequency range from 5 Hz to 12.5 Hz were calculated in every time range of 50 ms (hereinafter referred to as wavelet characteristic amounts).

In order to examine the arousal level and eye fatigue after an auditory event-related potential measurement, 7-leveled subjective reporting was asked to be made after the auditory event-related potential measurement experiment under each condition. By defining “very sleepy” as 1 and “not sleepy at all” as 7 for the arousal level, and by defining “very tired” as 1 and “not tired at all as 7” for the eye fatigue, each participant was asked to report his or her current state with a number. The reason for examining eye fatigue is to know whether any burden associated with the video viewing is on the eyes. Video viewing does not inherently belong in auditory stimulation measurement. The inventors preferred that the burden associated with video viewing should be minimized, and thus decided to examine eye fatigue.

Furthermore, subjective UCL value measurement was also conducted. Similarly to conventional studies (Takashi KIMITSUKI, et al., “Inner ear auditory testing in patients with normal hearing showing hyperacusis”, 2009, the subjective UCL value was measured by presenting discontinuous sounds with an ascending method using an audiometer, after which an unbearably loud sound pressure was asked to be reported. For each of three frequencies (1000, 2000, 4000 Hz) to be presented in the auditory event-related potential measurement experiment, measurement was taken for both ears, one ear at a time. In order to prevent anticipation of the sound pressure, the sound pressure at the start of the experiment was randomly selected from among 60, 65, and 70 dBHL. The sound pressure of the discontinuous sounds ascended by every 5 dB. An unbearably loud sound pressure was reported by raising a hand. Immediately after the participant raised a hand, the sound presentation was stopped, and the sound pressure was recorded as a subjective UCL value.

2-3. Results

2-3-1. Subjective Report (Arousal Level and Eye Fatigue)

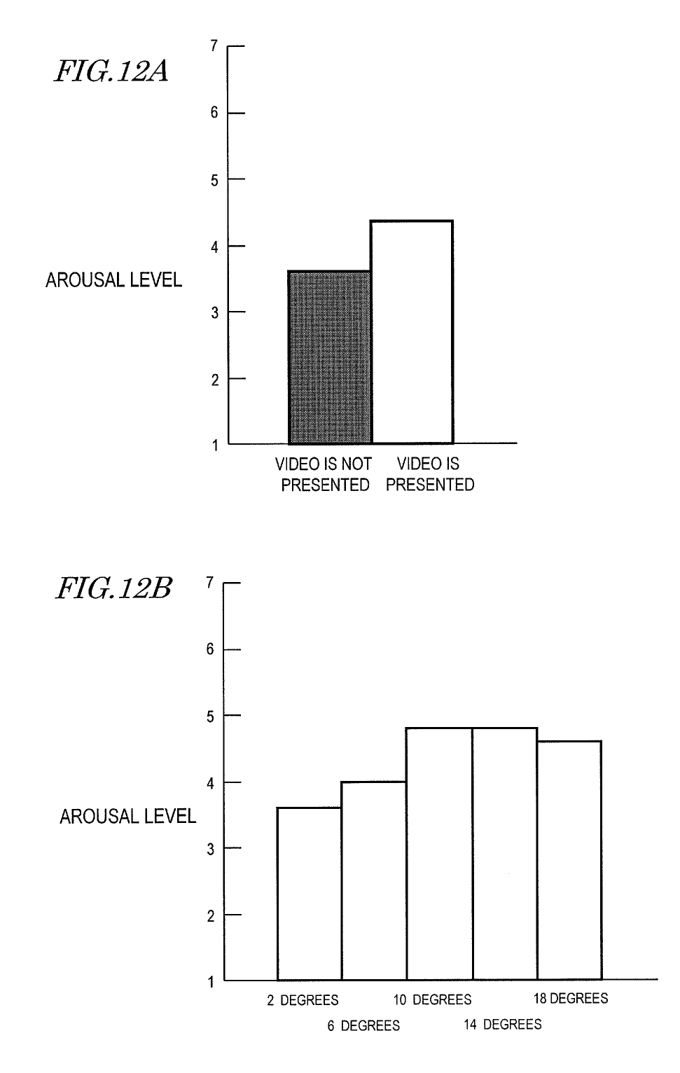

FIGS. 12A and 12B show results of subjective reporting of arousal levels in an experiment conducted after an electroencephalogram measurement under each condition. Each value represented by a bar in the chart is a mean value of subjectively reported arousal levels. In each of FIGS. 12A and 12B, the vertical axis represents the arousal level. As mentioned above, “very sleepy” corresponds to 1, and “not sleepy at all” corresponds to 7.

FIG. 12A shows a result of comparison between the no-video condition and the video-presented condition. It indicates that the arousal level is higher under the video-presented condition than under the no-video condition. Thus, it can be said that video presentation reduces a decrease in the arousal level during the auditory event-related potential measurement. FIG. 12B shows a mean value of arousal levels for each different size of video presentation, under the video-presented condition. It indicates that, while the video size is between 2 degrees and 10 degrees, the arousal level increases with an increase in video size. This partly agrees with conventional study results (Reeves, B. and Nass, C. (1996). The Media Equation: How people treat computers, television and new media like real people and places).

However, the arousal level no longer improves when the video size is larger than 10 degrees. This indicates that the effect of reducing a decrease in the arousal level by video presentation exhibits no difference once the video size becomes larger than 10 degrees.

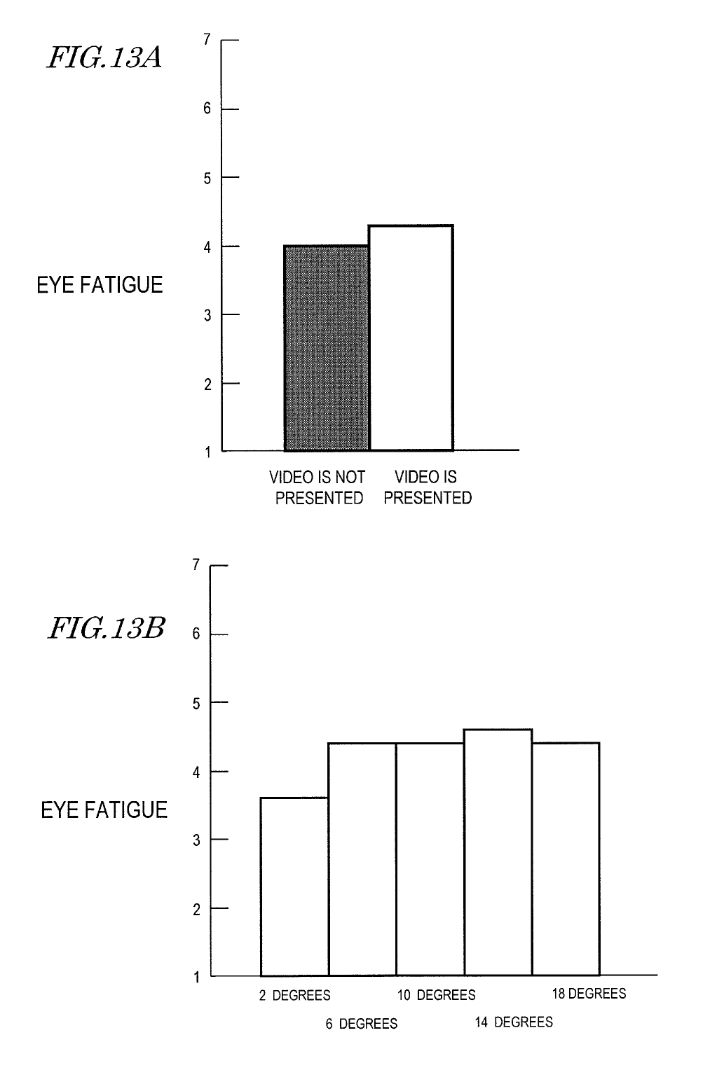

FIGS. 13A and 13B shows a mean value of subjectively reported eye fatigue. In FIGS. 13A and 13B, the vertical axis represents the arousal level, where “very tired” corresponds to 1 and “not tired at all” corresponds to 7, as mentioned above. FIG. 13A shows a comparison between the no-video condition and the video-presented condition. It indicates that there is less eye fatigue under the video-presented condition than under the no-video condition. Thus, it can be said that the eyes are less likely to be tired while watching video than while staring at a fixation point during the auditory event-related potential measurement. In everyday life, there are not many instances of staring at a fixation point with suppressed eye movement; this is the presumable reason why the eyes are likely to become tired even though the amount of eye movement itself may be small. FIG. 13B shows a mean value of eye fatigue for each different size of video presentation, under the video-presented condition. It indicates that large eye fatigue exists only under the video-at-2 degrees condition, unlike in any other condition. This is presumably because the size of the presented video is too small under the video-at-2 degrees condition, thus creating a situation which is similar to an instance of staring at a fixation point.

2-3-2. Electroencephalogram

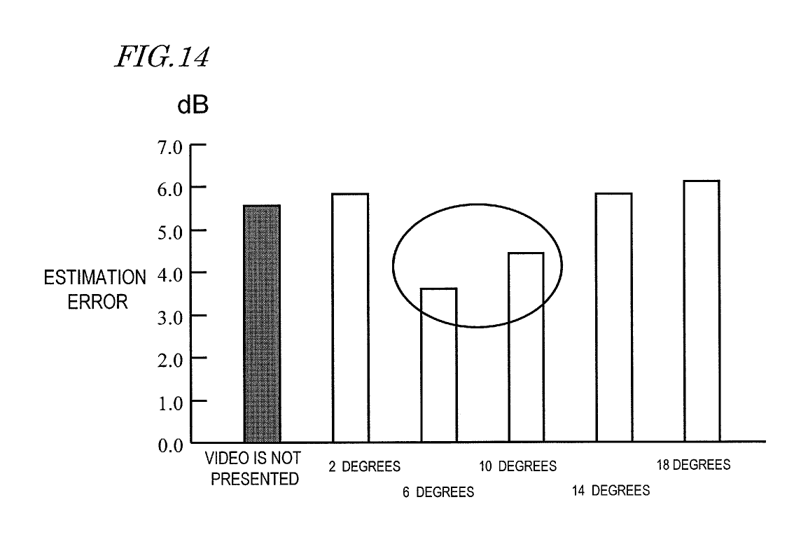

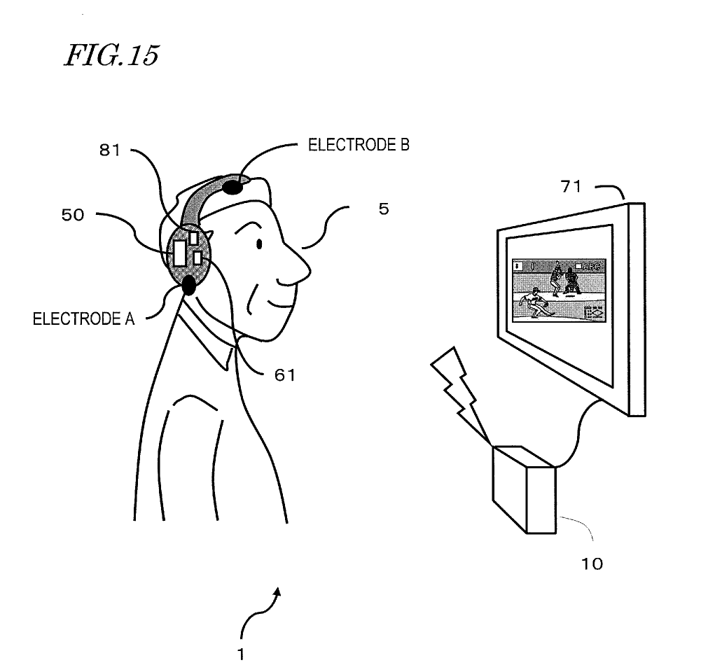

FIG. 14 shows estimation errors with different sizes of video presentation in an experiment conducted by the inventors. More specifically, for each different condition, FIG. 14 shows an average error between a subjective UCL value and an uncomfortable sound pressure for each participant and each frequency which is estimated by applying linear discrimination to auditory event-related potentials in response to auditory stimulations of sound pressures lower than a sound pressure which is generally evaluated to be the UCL. The vertical axis in FIG. 14 represents a mean value of estimation errors. Under the no-video condition, the estimation error had a mean value of 5.6 dB. Under the video-at-2 degrees condition across to the video-at-18 degrees condition, the estimation errors had mean values of 5.8 dB, 3.6 dB, 4.4 dB, 5.8 dB, and 6.1 dB, respectively. It can be seen that the mean value of estimation errors is smaller under the video-at-6 degrees condition and the video-at-10 degrees condition than under the no-video condition. Thus, it would be appropriate that the size of the video to be presented during the auditory event-related potential measurement is larger than a viewing angle of 2 degrees and smaller than a viewing angle of 14 degrees.

The reasons thereof will now be discussed. In a subjective report after the video-at-2 degrees condition, the arousal level was low and eye fatigue was high. This points to a possible reason for the increased estimation error under the video-at-2 degrees condition: a decrease in the arousal level. The reason why the estimation error increases when the video size is 14 degrees or greater is presumably that electrooculographic noise is mixed in the auditory event-related potential. As the video size increases, the electrooculographic noise mixed in the electroencephalogram increases substantially linearly because of an increased eye movement distance.

In any event, according to the aforementioned experiment conducted by the inventors, an auditory event-related potential measurement with an improved accuracy can be realized by presenting a video of a size which is defined by a viewing angle larger than 2 degrees and smaller than 14 degrees, simultaneously with auditory stimulations.

Hereinafter, the auditory event-related potential measurement system will be described in terms of illustrative embodiments according to the present disclosure.

<Outline of the Auditory Event-Related Potential Measurement System>

The auditory event-related potential measurement system according to the present embodiment presents a video in a size which is considered appropriate during the auditory event-related potential measurement, and realizes a highly accurate auditory event-related potential measurement which does not suffer much from fluctuations in the arousal level of the user and mixing of noise due to video watching.

In the present embodiment, by providing a probe electrode at the central portion (Cz) and a reference electrode at the right mastoid, an electroencephalogram is measured as a potential difference between the probe electrode and the reference electrode. Note that the level and polarity of a characteristic component of the event-related potential may possibly vary depending on the sites at which electrodes for electroencephalogram measurement are worn, and on the positions at which the reference electrode and the probe electrode are set. However, based on the following description, those skilled in the art should be able to extract a characteristic feature of the event-related potential and perform an auditory event-related potential measurement by making appropriate modifications in accordance with the particular reference electrode and probe electrode used. Such variants are encompassed within the present disclosure.

<Environment of Use>

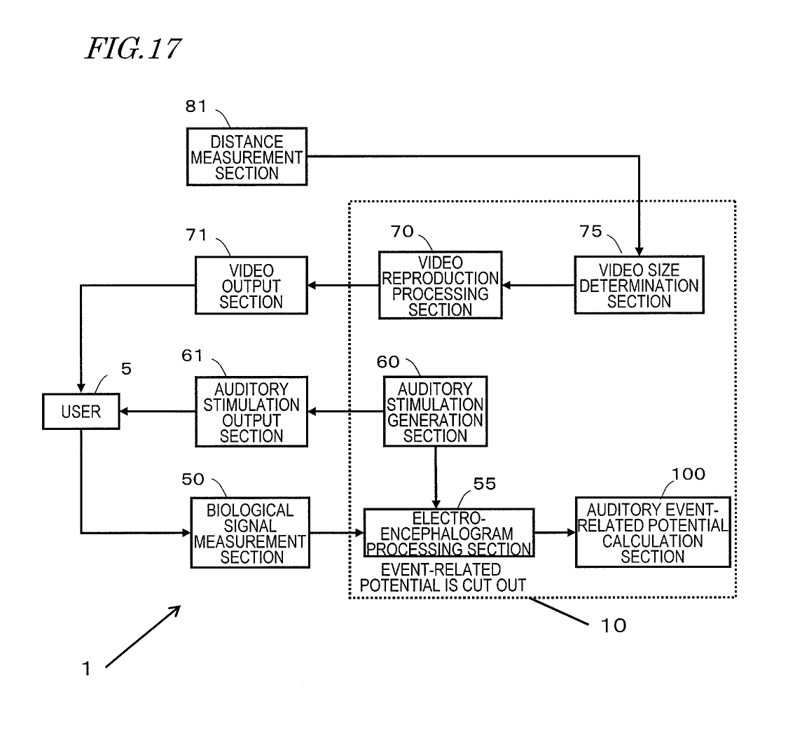

FIG. 15 shows a construction and an environment of use for an auditory event-related potential measurement system 1. The auditory event-related potential measurement system 1 (hereinafter referred to as the “measurement system 1”) is illustrated as an example corresponding to the system construction (FIG. 17) of Embodiment 1 described later.

The measurement system 1 measures an auditory event-related potential of a user 5 with a high accuracy. An electroencephalogram signal of the user 5 is acquired by a biological signal measurement section 50 which is worn on the head of the user 5, and is sent in a wired or wireless manner to an auditory event-related potential measurement apparatus 10 (hereinafter referred to as the “measurement apparatus 10”).

In a wired or wireless manner, an auditory stimulation output section 61 and a video output section 71 receive auditory stimulation information and video information, respectively, from the measurement apparatus 10, and present an auditory stimulation and a video, respectively, to the user 5. A distance measurement section 81 measures the distance between the eye position of the user 5 and the video output section 71, and sends the measurement result in a wired or wireless manner to the measurement apparatus 10. The measurement system 1 shown in FIG. 15 includes the biological signal measurement section 50 and the auditory stimulation output section 61 within the same housing; however, this is only an example. The biological signal measurement section 50 and the auditory stimulation output section 61 may be provided in separate housings.

The biological signal measurement section 50 is a measuring instrument which measures a biological signal of the user. In the present disclosure, one example of the biological signal measurement section 50 may be an electroencephalograph. The biological signal measurement section 50 is connected to at least two electrodes A and B. For example, electrode A is attached to a mastoid of the user 5, whereas electrode B is attached to a central portion (so-called Cz) on the scalp of the user 5. The biological signal measurement section 50 measures an electroencephalogram of the user 5 that corresponds to a potential difference between electrode A and electrode B, and outputs an electroencephalogram signal.

The auditory stimulation output section 61 is headphones or loudspeakers for outputting an auditory stimulation to the user 5, for example.

The video output section 71 is a monitor for presenting a video to the user 5, for example.

The distance measurement section 81 is a range finder which measures the distance between the eye position of the user 5 and the video output section 71 at predetermined timing. Any technique may be used so long as the distance between the eye position of the user 5 and the video output section 71 can be measured. For example, a reflected wave of an ultrasonic wave or a millimeter wave may be used.

In accordance with the distance between the user 5 and the video output section 71 as received from the distance measurement section 81, the measurement apparatus 10 calculates video an appropriate size for the video, and while presenting a video, e.g., a movie or a TV program in that size to the user 5, presents auditory stimulations, and measures auditory event-related potentials.

<Hardware Construction>

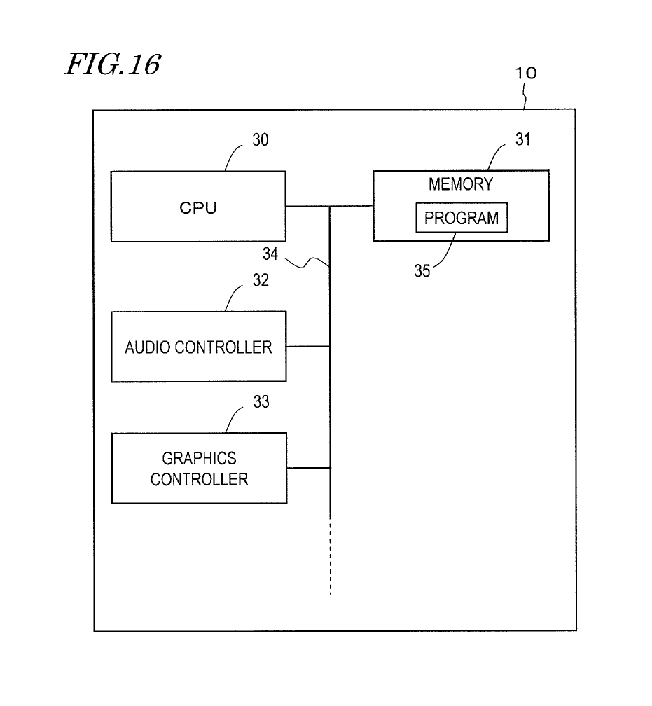

FIG. 16 shows the hardware construction of the measurement apparatus 10 of the present embodiment. The measurement apparatus 10 includes a CPU 30, a memory 31, an audio controller 32, and a graphics controller 33. The CPU 30, the memory 31, the audio controller 32, and the graphics controller 33 are connected to one another via a bus 34, so that data exchange among them is possible.

The CPU 30 executes a computer program 35 which is stored in the memory 31. A processing procedure which is illustrated by a subsequently-described flowchart is described in the computer program 35. In accordance with the computer program 35, the measurement apparatus 10 performs a process of controlling the entire measurement system 1, e.g., auditory stimulation generation, video reproduction, detection of luminance changes in video, and determination of ignorable trials. This process will be described in detail later.

In accordance with an instruction from the CPU 30, the audio controller 32 outputs via the auditory stimulation output section 61 auditory stimulations to be presented, each at a designated timing and with a designated sound pressure and duration.

In accordance with an instruction from the CPU 30, the graphics controller 33 outputs a video via the video output section 71.

Note that the measurement apparatus 10 may be implemented as a piece of hardware (e.g., a DSP) consisting of a semiconductor circuit having a computer program therein. Such a DSP can realize all functions of the aforementioned CPU 30, memory 31, audio controller 32, and graphics controller 33 on a single integrated circuit.

The aforementioned computer program 35 may be distributed on the market in the form of a product recorded on a storage medium such as a CD-ROM, or transmitted through telecommunication lines such as the Internet.

Upon reading the computer program 35, a device having the hardware shown in FIG. 16 (e.g., a PC) is able to function as the measurement apparatus 10 of the present embodiment.

<Construction of the Measurement System 1>

FIG. 17 shows the functional block construction of the measurement system 1 of the present embodiment. The measurement system 1 includes the biological signal measurement section 50, the auditory stimulation output section 61, the video output section 71, a distance measurement section 81, and the measurement apparatus 10. The component elements of the measurement system 1 are interconnected in a wired or wireless manner. The user 5 block is illustrated for ease of description.

FIG. 17 also shows detailed functional blocks of the measurement apparatus 10. The measurement apparatus 10 includes an electroencephalogram processing section 55, an auditory stimulation generation section 60, a video reproduction processing section 70, a video size determination section 75, and an auditory event-related potential calculation section 100.

The respective functional blocks of measurement apparatus 10 correspond to functions which are occasionally realized by the CPU 30, the memory 31, the audio controller 32, and the graphics controller 33 as a whole when the program described in connection with FIG. 16 is executed.

Hereinafter, the component elements of the measurement system 1 will be described.

<Auditory Stimulation Generation Section 60>

The auditory stimulation generation section 60 determines information of an auditory stimulation to be presented to the user 5. The auditory stimulation information includes which of the right or left ear of the user 5 the auditory stimulation is to be presented to, and the frequency and sound pressure of the auditory stimulation to be presented. The sound pressure of the auditory stimulation to be presented is determined within a range of sound pressures lower than a sound pressure which is generally evaluated to be the UCL, for example. The frequency of the auditory stimulation to be presented and the right or left ear may be randomly determined under the following constraints, for example.

No auditory stimulation of the same frequency as an immediately previous auditory stimulation is selected.

The right or left ear is selected in a random order.

However, not more than four auditory stimulations are presented successively to either the right or left ear. By doing so, the influence of taming (habituation) of the electroencephalogram due to successive presentation of auditory stimulations to the same ear and at the same frequency is reduced, whereby a highly accurate auditory event-related potential measurement is realized.

The auditory stimulation generation section 60 generates an audio signal of the determined auditory stimulation, and sends it to the auditory stimulation output section 61 with a predetermined stimulation interval. The auditory stimulation may be a toneburst sound having a rise and fall of 3 ms, for example. The duration of an auditory stimulation is set to be e.g. 25 ms or more, so that an auditory event-related potential will be stably induced. The predetermined stimulation interval is set to a time which is equal to or greater than the duration of the auditory stimulation but equal to or less than 2 seconds. For example, it may be 500 ms, or 1 second.

At the timing of sending auditory stimulation information to the auditory stimulation output section 61, the auditory stimulation generation section 60 outputs a trigger to the electroencephalogram processing section 55. This trigger is used when cutting out an event-related potential in response to an auditory stimulation at the electroencephalogram processing section 55. Moreover, at the timing of sending auditory stimulation information to the auditory stimulation output section 61, the auditory stimulation generation section 60 sends information of the timing of presenting the auditory stimulation, the right or left ear, and the frequency and sound pressure of the auditory stimulation to the electroencephalogram processing section 55.

Note that the auditory stimulation generation section 60 may be composed of an input section, such that information which is input via the input section by the user 5 or a person who tests the hearing of the user 5 is utilized as the auditory stimulation information. In other words, in the measurement system 1, auditory stimulations may be externally received, rather than being internally generated.

<Auditory Stimulation Output Section 61>

The auditory stimulation output section 61 is connected to the auditory stimulation generation section 60 in a wired or wireless manner. The auditory stimulation output section 61 reproduces auditory stimulation data which is generated by the auditory stimulation generation section 60, and presents it to the user 5. With the auditory stimulation presentation to the user 5 as a trigger, the auditory stimulation output section 61 may send information of the point in time at which the auditory stimulation was presented, to the electroencephalogram processing section 55.

<Biological Signal Measurement Section 50>

The biological signal measurement section 50 measures a biological signal of the user 5. As the biological signal, the biological signal measurement section 50 measures an electroencephalogram signal which corresponds to a potential difference between the probe electrode and the reference electrode. Frequency filtering with an appropriate cutoff frequency may be applied to the electroencephalogram signal. The biological signal measurement section 50 sends the electroencephalogram signal as measured or the filtered electroencephalogram signal to the electroencephalogram processing section 55. Hereinafter, a measured electroencephalogram signal or a filtered electroencephalogram signal may be referred to as electroencephalogram data.

In the case where a band-pass filter is used as the frequency filter, the cutoff frequency may be set so as to pass e.g. 5 Hz to 15 Hz. It is assumed that the user 5 has worn the electroencephalograph in advance. The probe electrode for electroencephalogram measurement is attached at the central portion Cz, for example.

<Electroencephalogram Processing Section 55>

From the electroencephalogram data received from the biological signal measurement section 50, the electroencephalogram processing section 55 acquires an event-related potential in a predetermined zone, based on the trigger received from the auditory stimulation generation section 60 or the auditory stimulation output section 61 as a starting point. For example, the electroencephalogram processing section 55 cuts out an event-related potential in a zone from 100 ms before the auditory stimulation presentation to 400 ms after the auditory stimulation presentation.

The zone to cut out may be any zone that contains a targeted component of the auditory event-related potential. For instance, a positive component (P1 component) appearing in a zone from 50 ms to 150 ms based on a point of auditory stimulation will be taken as an example. The zone to cut out may be a zone from 100 ms before the auditory stimulation presentation to 400 ms after the auditory stimulation presentation as mentioned above, or may be a zone from 50 ms to 150 ms based on the point of auditory stimulation. The electroencephalogram processing section 55 sends the cutout event-related potential to the auditory event-related potential calculation section 100.

Note that a “cutout event-related potential” does not only mean a piece of electroencephalogram data which has actually been extracted from a predetermined zone of a measured electroencephalogram signal, but also encompasses a piece of electroencephalogram data containing the necessary potential in an extractable state, which does not need to have actually been extracted. For example, a necessary event-related potential is ready extractable so long as there are the electroencephalogram signal and zone information identifying a predetermined zone within that electroencephalogram signal. It can be said that, by acquiring these, the electroencephalogram processing section 55 is able to obtain a “cutout event-related potential”.

<Distance Measurement Section 81>

The distance measurement section 81 is a range finder which measures the distance between the eye position of the user 5 and the video output section 71 at a predetermined timing. Any technique may be used so long as the distance between the eye position of the user 5 and the video output section 71 can be measured. For example, a reflected wave of an ultrasonic wave or a millimeter wave may be used. Then, the measured result is sent to the video size determination section 75.

Preferably, the distance measurement section 81 measures an angle between the eye position of the user 5 and the video output section 71, e.g., the angle between a line segment connecting the eye position of the user 5 and a center of the video which is output by the video output section 71, and a line segment which is perpendicular to the screen of the video output section 71. The distance measurement section 81 sends the measured angle to the video size determination section 75.

<Video Size Determination Section 75>

Based on the distance between the user 5 and the video output section 71 as received from the distance measurement section 81, the video size determination section 75 determines the size of the video to be presented to the user in a range which is greater than a viewing angle of 2 degrees and smaller than a viewing angle of 14 degrees, by using eq. 1 above. Preferably, the video size determination section 75 determines the video size to a viewing angle of equal to or greater than 6 degrees but equal to or less than 10 degrees.

For example, in the case where the distance between the user 5 and the video output section 71 is 1 m, the diagonal length of the video is to be determined within a range of no less than 3.5 cm and no more than 24.9 cm. Then, the determined video size is sent to the video reproduction processing section 70.

Moreover, based on the information of the angle between the eye position of the user 5 and the video output section 71, and the distance between the user 5 and the video output section 71, the video size may be determined in a range which is greater than a viewing angle of 2 degrees and smaller than a viewing angle of 14 degrees. In this case, based on the information of the angle between the eye position of the user 5 and video output section 71, initial positioning is adjusted, and then the video size is determined based on eq. 1.

<Video Reproduction Processing Section 70>

In a hard disk drive not shown, for example, the video reproduction processing section 70 previously retains data of a video (content) to be presented to the user. The video reproduction processing section 70 reproduces the video in the video size which is received from the video size determination section 75. In other words, the video reproduction processing section 70 controls outputting of the video content.

A video content is information containing a chronological sequence of a plurality of at least partially differing images: for example, a movie, or a TV program such as a drama or sport broadcasting. For the purpose of suppressing fluctuations in the arousal level of the user 5, the user 5 may be allowed to select a content according to the level of interest of the user 5.

The present embodiment assumes that the video content does not contain any audio information. However, the video content may contain some audio information, in which case the audio information contained in the video content may be prohibited from being output by, for example, the video reproduction processing section 70 exerting control for not allowing the audio to be output through the loudspeakers.

<Video Output Section 71>

The video output section 71 is connected to the video reproduction processing section 70 in a wired or wireless manner, and outputs a video which has been subjected to a reproduction process by the video reproduction processing section 70. It is assumed that the video is always being reproduced during the auditory event-related potential measurement.

<Auditory Event-Related Potential Calculation Section 100>

The auditory event-related potential calculation section 100 (hereinafter referred to as “the calculation section 100”) takes an arithmetic mean of the event-related potentials received from the electroencephalogram processing section 55, based on the auditory stimulation information received from the auditory stimulation generation section 60. The arithmetic mean may be taken for each of the right or left ear, each frequency, and each sound pressure.

Since the event-related potential is a very minute potential (e.g., several μV), it is commonplace to take an arithmetic mean of measured event-related potentials. However, in the case where accurate acquisition of an event-related potential is possible, an event-related potential in response to a single sound may be used. In this case, the calculation section 100 can be omitted.

<Processing by the Measurement System 1>

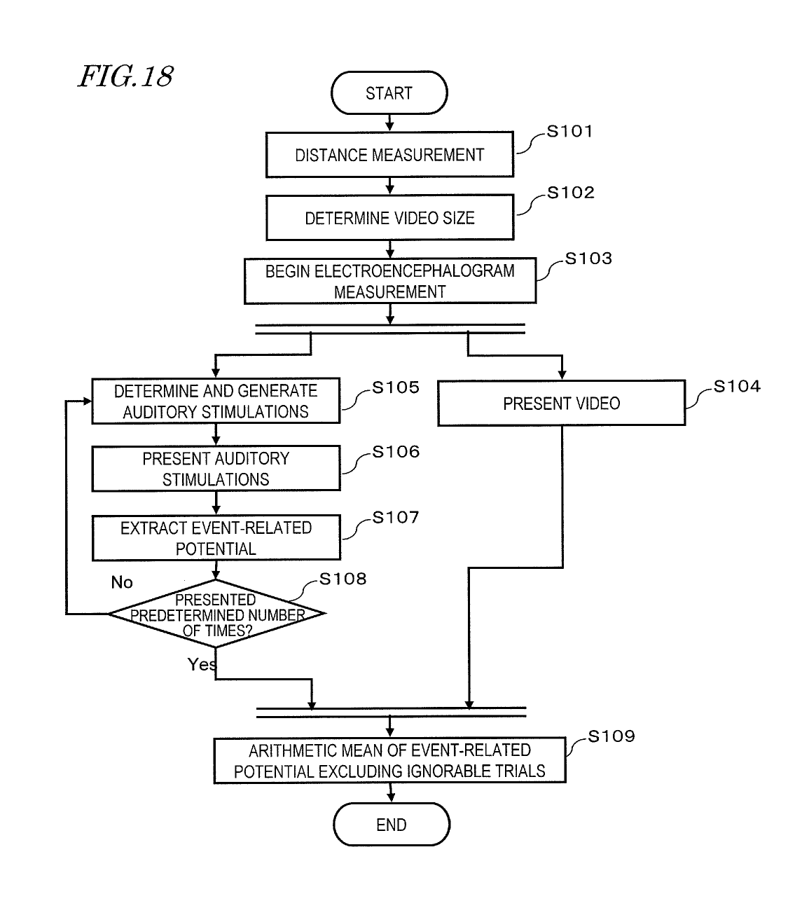

Next, with reference to FIG. 18, a processing procedure performed by the measurement system 1 in FIG. 17 will be described. FIG. 18 is a flowchart showing a procedure of processing performed by the measurement system 1.

At step S101, the distance measurement section 81, which is a range finder, measures the distance between the eye position of the user 5 and the video output section 71. Any technique may be used so long as the distance between the eye position of the user 5 and the video output section 71 can be measured. For example, a reflected wave of an ultrasonic wave or a millimeter wave may be used. Then, the measured result is sent to the video size determination section 75.

At step S102, based on the distance between the user 5 and the video output section 71 as received from the distance measurement section 81, the video size determination section 75 determines the size of the video to be presented to the user in a range which is greater than a viewing angle of 2 degrees and smaller than a viewing angle of 14 degrees, by using eq. 1 above. For example, in the case where the distance between the user 5 and the video output section 71 is 1 m, the diagonal length of the video is to be determined within a range of no less than 3.5 cm and no more than 24.9 cm. Then, the determined video size is sent to the video reproduction processing section 70.

At step S103, the biological signal measurement section 50 measures an electroencephalogram of the user 5 as a biological signal. Then, the biological signal measurement section 50 applies frequency filtering with an appropriate cutoff frequency to the electroencephalogram data, and sends continuous electroencephalogram data to the electroencephalogram processing section.

At step S104, in a size as determined by the video size determination section 75, the video reproduction processing section 70 reproduces a video content which is previously-retained in the video reproduction processing section 70, and presents it to the user 5 via the video output section 71. The video content may be a movie, or a TV program such as a drama or sport broadcasting, for example. For the purpose of suppressing fluctuations in the arousal level of the user 5, the user 5 may be allowed to select a content according to the level of interest of the user 5. The present embodiment assumes that the video content is presented with no sounds.

At step S105, the auditory stimulation generation section 60 determines information of an auditory stimulation to be presented to the user 5. The auditory stimulation information includes which of the right or left ear of the user 5 the auditory stimulation is to be presented to, and the frequency and sound pressure of the auditory stimulation to be presented. The sound pressure of the auditory stimulation is determined within a range of sound pressures lower than a sound pressure which is generally evaluated to be the UCL. Then, the auditory stimulation generation section 60 determines the auditory stimulation as determined, and sends it to the auditory stimulation output section 61 with a predetermined stimulation interval. At the timing of sending auditory stimulation information to the auditory stimulation output section 61, the auditory stimulation generation section 60 outputs a trigger to the electroencephalogram processing section 55. Moreover, at the timing of sending auditory stimulation information to the auditory stimulation output section 61, the auditory stimulation generation section 60 sends information of the timing of presenting the auditory stimulation, the right or left ear, and the frequency and sound pressure of the auditory stimulation to the electroencephalogram processing section 55.

At step S106, the auditory stimulation output section 61 reproduces auditory stimulation data which is generated by the auditory stimulation generation section 60, and presents it to the user 5.

At step S107, from the electroencephalogram data received from the biological signal measurement section 50, the electroencephalogram processing section 55 cuts out an event-related potential in a predetermined zone (e.g., a zone from 100 ms before the auditory stimulation presentation to 400 ms after the auditory stimulation presentation), based on the trigger received from the auditory stimulation generation section 60 as a starting point. Then, the electroencephalogram processing section 55 sends the event-related potential to the calculation section 100. Moreover, the electroencephalogram processing section 55 sends the information of the right or left ear, frequency, and sound pressure of the auditory stimulation as received from the auditory stimulation generation section 60 to the calculation section 100.

Step S108 is a branching based on whether the auditory stimulation presentation and event-related potential extraction at steps S105 to S107 has been performed a predetermined number of times, which is previously set. For example, assuming that 30 times of repetition are made at three sound pressures for each of five frequencies with respect to each of the right and left ears, the predetermined number of times is 900 times (2×5×3×30). If Yes at step S108, control proceeds to step S109; if No, control returns to step S105 to repeat the auditory stimulation presentation and event-related potential extraction.

At step S109, based on the auditory stimulation information received from the electroencephalogram processing section 55, the calculation section 100 takes an arithmetic mean of event-related potentials also received from the electroencephalogram processing section 55. In the present disclosure, step S109 is not essential because, through the processes from steps S101 to S108 including video presentation, the user 5 has received auditory stimulations at a relatively high arousal level, and the auditory event-related potentials evoked by the auditory stimulations have a high accuracy. It must be noted that the process of step S109 is introduced for a further enhanced accuracy.

With the measurement system 1 of the present embodiment, during an auditory event-related potential measurement, a video is presented in a size whose viewing angle between diagonal corners is in a range greater than 2 degrees and smaller than 14 degrees, in accordance with the distance between the user and the display. Thus, a highly accurate auditory event-related potential measurement which is not susceptible to the influence of fluctuations in the arousal level of the user and any electrooculographic noise mixed due to video watching.

In the present specification, the video size determination section 75 calculates a viewing angle according to eq. 1 by relying on the diagonal length of the presented video being S. In other words, in determining the video size, the video size determination section 75 deems the entire video displaying region as the range (region) in which a gaze movement may possibly occur; however, this is only an example. If it is possible to previously identify a range (partial region) within the video in which a gaze movement may occur, the diagonal length of that partial region may be defined as S.

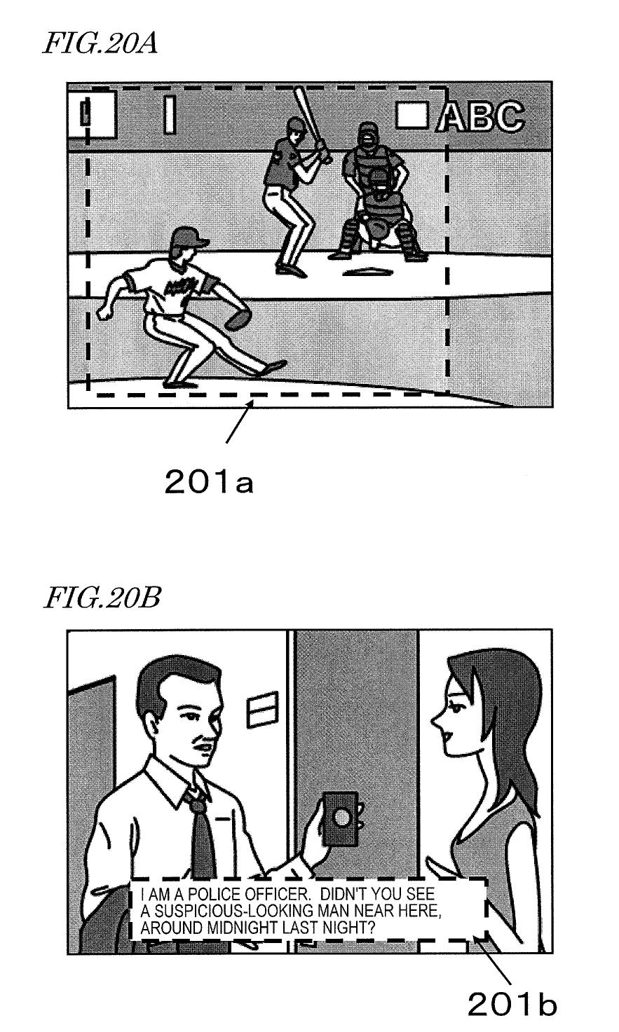

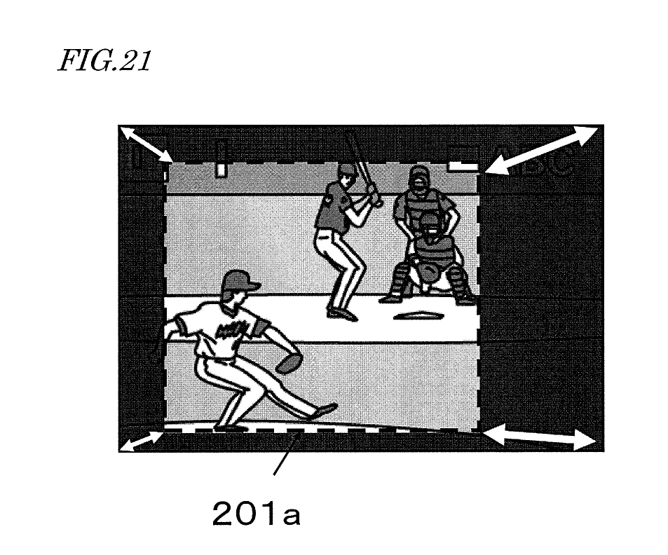

For example, FIGS. 20A and 20B each show a region which may define a diagonal length with a broken line. As shown in FIG. 20A, the diagonal length of a main region 201a of the content may be defined as S, or as shown in FIG. 20B, the diagonal length of a subtitle displaying region 201b may be defined as S. Note that the diagonal length of the main region of a given content or a subtitle displaying region may be previously retained in a database, or calculated in real time. In this case, the video size determination section 75 does not need to determine the size of the video per se, but may determine the size of any such region that defines the diagonal length S. FIG. 21 schematically shows a main region 201a whose size is changeable. For example, as shown in FIG. 21, the video size determination section 75 may alter the size of the main region so that the main region of the content accounts for a part of the entire video. In doing so, the region excluding the main region may be grayed out.

Alternatively, the video size may be kept constant, but the position of the user 5 or the video output section 71 may be adjusted to ensure that the distance between the eye position of the user 5 and the video output section 71 has a predetermined value such that the viewing angle between diagonal corners is greater than 2 degrees and smaller than 14 degrees. In this case, the distance measurement section and the video size determination section 75 may be omitted.

Note that distance measurement may be conducted at predetermined intervals, and the video size may be redefined during the auditory event-related potential measurement. In that case, the size of the entire video may be dynamically changed, or, in the case where the region excluding the main region is grayed out as shown in FIG. 21, the size of the grayed-out region may be changed as indicated by arrows, without changing the size of the entire video.

In the case where the viewing angle between diagonal corners is in a range greater than 2 degrees and smaller than 14 degrees, the video size may be determined according to the genre of the video to be reproduced. For example, in a sport broadcasting which is expected to cause frequent eye movements, the viewing angle between diagonal corners may be set small, e.g., greater than 2 degrees and smaller than 8 degrees; for a drama which is expected not to cause frequent eye movements, the viewing angle between diagonal corners may be 8 degrees or more but smaller than 14 degrees.

Although the present embodiment does not accumulate results of auditory event-related potential measurement, a database for result accumulation may be additionally provided to accumulate results.

The present embodiment assumes that the video to be presented is previously retained in the video reproduction processing section 70. Alternatively, a TV video which is being broadcast in real time during the auditory event-related potential measurement may be presented. In that case, too, luminance changes in the TV video may be detected by the luminance change detection section 76 in a similar manner.

When measuring a UCL of the user 5, a P1 component of the user 5 is acquired. When the P1 component is equal to or greater than a predetermined threshold value, it means that the user 5 perceives the sound pressure of the presented sound (auditory stimulation) to be loud. For each frequency, etc., a sound pressure which is felt loud to be the user 5 is measured, and based on the measured information, a hearing aid can be adjusted.

Note that the measurement apparatus 10 at least includes the video reproduction processing section 70 and the electroencephalogram processing section 55.

The above embodiment illustrates that a presumably appropriate video size exists when the viewing angle between diagonal corners of the video is in a range greater than 2 degrees and smaller than 14 degrees. This range is merely a range which was derived from experimental event-related potential waveforms obtained by the inventors. It is expected that this range may vary under any condition which differs from the condition of the experimentation by the inventors, concerning the type of presented video, the physical conditions of the experimental participants on the day, differences in vision, and so on. It is conceivable that a value which is 2 degrees or less (e.g. 1.5 degrees) may be the lower limit, and a value which is 14 degrees or more (e.g. 14.5 degrees) may be the upper limit. This range may be varied so long as the arousal level of the user is sustainable under the particular condition that the auditory event-related potential measurement system is used. The expression “range greater than 2 degrees and smaller than 14 degrees” as used in the present specification is to be interpreted as not exclusive, but rather inclusive, of any such variation.

With the auditory event-related potential measurement apparatus according to the present disclosure and the auditory event-related potential measurement system incorporating the auditory event-related potential measurement apparatus, a video is presented concurrently with auditory stimulations in a size which is considered appropriate, whereby a highly accurate auditory event-related potential measurement is realized while reducing a decrease in the arousal level of the user and suppressing the influence of noise mixed due to video watching. Results of the highly accurate auditory event-related potential measurement can be used in an objective hearing evaluation of the user.

While the present invention has been described with respect to exemplary embodiments thereof, it will be apparent to those skilled in the art that the disclosed invention may be modified in numerous ways and may assume many embodiments other than those specifically described above. Accordingly, it is intended by the appended claims to cover all modifications of the invention that fall within the true spirit and scope of the invention.

What is claimed is:

1. An auditory event-related potential measurement system comprising:
a size determination section configured to determine a size of a region within a video to be presented to a user so that the region has a viewing angle between diagonal corners in a range greater than 2 degrees and smaller than 14 degrees;
a video output section configured to present to the user a video including a region of the size determined by the size determination section;
an auditory stimulation output section configured to present an auditory stimulation to the user during a period in which the video is being presented to the user;
a biological signal measurement section configured to measure an electroencephalogram signal of the user; and
an electroencephalogram processing section configured to acquire an event-related potential from the electroencephalogram signal as reckoned from a point in time at which the auditory stimulation is presented.

2. The auditory event-related potential measurement system of claim 1, further comprising a calculation section configured to take an arithmetic mean of the event-related potential acquired by the electroencephalogram processing section.

3. The auditory event-related potential measurement system claim 1, further comprising a distance measurement section configured to measure a distance from an eye position of the user to the video output section, wherein
the size determination section determines the size of the region within the video based on the distance.

4. The auditory event-related potential measurement system of claim 3, wherein,
the distance measurement section measures the distance at a predetermined timing; and
based on the measured distance, the size determination section changes the size of the region within the video while the event-related potential is being measured.

5. The auditory event-related potential measurement system of claim 4, further comprising a video reproduction processing section configured to retain at least one type of video content to be presented to the user, and configured to perform a reproduction process of a retained video content.

6. The auditory event-related potential measurement system of claim 5, wherein the video content does not contain audio information.

7. The auditory event-related potential measurement system of claim 5, wherein, when the video content contains any audio information, the video output section prohibits outputting of the audio.

8. The auditory event-related potential measurement system of claim 5, wherein,
the video reproduction processing section retains a plurality of types of video contents; and
the video reproduction processing section performs a reproduction process of a video content selected by the user from among the plurality of types of video contents.

9. The auditory event-related potential measurement system of claim 1, further comprising an auditory stimulation generation section configured to determine which of right and left ears of the user the auditory stimulation is to be presented to, configured to determine a frequency and a sound pressure of the auditory stimulation, and configured to generate the auditory stimulation with characteristics so determined.

10. The auditory event-related potential measurement system of claim 1, wherein the size determination section determines the size of the video so that a viewing angle between diagonal corners of the entire video presented to the user is in a range greater than 2 degrees and smaller than 14 degrees.

11. The auditory event-related potential measurement system of claim 1, wherein the size determination section determines the size of a partial region within the video so that a viewing angle between diagonal corners of the partial region within the video presented to the user is in a range greater than 2 degrees and smaller than 14 degrees.

12. An auditory event-related potential measurement method comprising:
determining a size of a region within a video to be presented to a user so that the region has a viewing angle between diagonal corners in a range greater than 2 degrees and smaller than 14 degrees;
presenting to the user a video including a region of the size determined by the step of determining the size;
presenting an auditory stimulation to the user during a period in which the video is being presented to the user;
measuring an electroencephalogram signal of the user; and
acquiring an event-related potential from the electroencephalogram signal as reckoned from a point in time at which the auditory stimulation is presented.

13. A computer program stored on a non-transitory computer-readable medium, and to be executed by a computer provided in an auditory event-related potential measurement apparatus of an auditory event-related potential measurement system,
the computer program causing the computer to execute:
determining a size of a region within a video to be presented to a user so that the region has a viewing angle between diagonal corners in a range greater than 2 degrees and smaller than 14 degrees;
presenting to the user a video including a region of the size determined by the step of determining the size;
presenting an auditory stimulation to the user during a period in which the video is being presented to the user;
acquiring an electroencephalogram signal of the user; and
acquiring an event-related potential from the electroencephalogram signal as reckoned from a point in time at which the auditory stimulation is presented.

14. An auditory event-related potential measurement apparatus comprising:
a size determination section configured to determine a size of a region within a video to be presented to a user so that the region has a viewing angle between diagonal corners in a range greater than 2 degrees and smaller than 14 degrees; and
an electroencephalogram processing section configured to acquire an event-related potential from an electroencephalogram signal measured by a biological signal measurement section, wherein,
when an auditory stimulation output section presents an auditory stimulation to the user during a period in which a video output section is presenting to the user a video including a region of the size determined by the size determination section, the electroencephalogram processing section acquires an event-related potential from the electroencephalogram signal as reckoned from a point in time at which the auditory stimulation is presented.
